(19) United States
(12) Patent Application Publication
(10) Pub. No.: US 2011/0129508 A1
(43) Pub. Date: Jun. 2, 2011

(54) METHODS AND COMPOSITIONS FOR THE MANAGEMENT OF PAIN USING OMEGA-CONOTOXINS

(75) Inventors: Ian Cooke, Hawthorn (AU); Colin Stanley Goodchild, Malvern (AU)

(73) Assignee: RELEVARE AUST. PTY LTD, Abbotsford (AU)

(21) Appl. No.: 12/991,373

(22) PCT Filed: May 6, 2009
(86) PCT No.: PCT/AU09/00563

§ 371 Date: Jan. 31, 2011

Related U.S. Application Data
(60) Provisional application No. 61/050,869, filed on May 6, 2008.

(51) Int. Cl. A61K 38/17; A61K 9/00; A61P 29/00; A61K 38/04

(52) U.S. Cl. 424/400; 514/18.3

(57) ABSTRACT

The present invention relates to the management of pain (nociceptive, neuropathic, inflammatory and disease related pains), using omega-conotoxins alone or in combination with neuronal excitation inhibitors (analgesics). The invention in particular provides methods, protocols, compositions and devices which treat, alleviate, prevent, diminish or otherwise ameliorate the sensation of pain.

### FILING DATA

This application is associated with and claims priority from U.S. Patent Application No. 61/050,869 filed on 6 May, 2008, the contents of which are incorporated by reference.

### FIELD

The present invention relates generally to the field of pain management, and in particular, the management of nociceptive and neuropathic pain. More particularly, the present invention provides methods, protocols, compositions and devices which treat, alleviate, prevent, diminish or otherwise ameliorate the sensation of pain.

### BACKGROUND

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Bibliographic details of the publications referred to by author in this specification are collected alphabetically at the end of the description.

Pain is a sensory experience associated with actual or potential tissue damage. Pain of any type is the most frequent reason for physician consultation in the United States, prompting half of all Americans to seek medical care annually. It is a major symptom in many medical conditions, significantly interfering with a person's quality of life and general functioning. Diagnosis is based on characterizing pain in various ways, according to duration, intensity, type (dull, burning or stabbing), source or location in body. Acute pain generally stops without treatment or responds to simple measures such as resting or taking an analgesic. However, if it persists and becomes intractable it then becomes chronic pain, in which pain is no longer considered a symptom but an illness by itself.

Of the different pain types, the management of nociceptive and neuropathic pain has been difficult. Stimulation of a nociceptor due to a chemical, thermal or mechanical event that has the potential to damage body tissue leads to nociceptive pain. Damage to a pain nerve itself leads to neuropathic pain.

Although there are numerous therapies available for nociceptor-induced pain, such as treatment with opioid and non-steroidal anti-inflammatory drugs (NSAIDs), these therapies are often unsatisfactory when administration is required over extended time frames due to the emergence of tolerance and adverse side-effects. For example, common side effects of treatment with opioids include constipation, nausea, sedation, respiratory depression, mycolonus, urinary retention, confusion, hallucinations and dizziness. In addition, extended administration typically leads to drug tolerance, resulting in the need for increased levels of drugs to be administered, thereby further exacerbating the side effects of the drugs.

In addition, treatment of neuropathic pain has not met with particular success. This is due to the distinct pathophysiochemical mechanisms and clinical manifestations associated with neuropathic pain relative to pain caused as a result of nociceptor stimulation. Agents useful in the treatment of pain caused as a result of nociceptor stimulation have reduced effectiveness in neuropathic pain treatment. In particular, the effectiveness of opioids in the treatment of neuropathic pain is diminished relative to their use in the treatment of pain caused as a result of nociceptor stimulation. In particular, drug dose response curves for treatment of neuropathic pain are shifted to the right of those for treatment of pain caused as a result of nociceptor stimulation or acute pain.

Accordingly, there is a need to develop safe and efficacious therapies for the short and long term treatment of nociceptive and neuropathic pain.

### SUMMARY

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Methods and compositions for treating, alleviating, preventing, diminishing or otherwise ameliorating the symptoms associated with pain and in particular the sensation of pain in a subject are provided. By “symptoms” is meant the perception or sensation of or the physical effects of pain. Reference to “pain” includes nociceptive pain and neuropathic pain as well as pain caused by disease conditions such as cancer and inflammation. The later types of pain are referred to herein as “cancer pain” and “inflammatory pain”. In particular, a method is contemplated for inducing an analgesic response to pain in a subject comprising the systemic, non-intrathecal administration to the subject of an amount of an omega conotoxin, either alone or in combination with a neuronal excitation inhibitor, which administration is effective at reducing the level of or otherwise ameliorating the sensation of pain. In a particular embodiment, the analgesic response does not induce sedation, including overt sedation.

Accordingly, one aspect of the present invention contemplates a method for inducing an analgesic response to pain in a subject, the method comprising the systemic, non-intrathecal administration to the subject of an amount of an omega conotoxin which is effective at reducing the level of or otherwise ameliorating the sensation of pain.

Another aspect of the present invention provides a method for inducing an analgesic response to pain in a subject, the method comprising the systemic, non-intrathecal administration to the subject of an amount of an omega conotoxin and a neuronal excitation inhibitor which is effective at reducing the level of or otherwise ameliorating the sensation of pain whilst not inducing overt sedation.

The pain may be nociceptive pain or neuropathic pain such as inflammatory pain or cancer pain.

Generally, the administration of the omega conotoxin alone or in combination with a neuronal excitation inhibitor does not induce or cause overt sedation. As used herein, an “omega conotoxin” or “ωo-conotoxin” is an N-type calcium channel antagonist which blocks the influx of calcium ions through a channel. In certain embodiments, the omega conotoxin, used either alone or in combination with a neuronal excitation inhibitor, is selected from the group consisting of CVID (also known as AM336 and leconotide), GVIA, MVIIA (also known as Ziconotide and Prialt) and SNX-111. The omega conotoxins contemplated for use in the methods or compositions of the present invention are also described in PCT Application No, PCT/AU99/00288, the contents of which are incorporated herein by reference.

In another aspect, the present invention is directed to a method for inducing an analgesic response in a subject suffering from pain, the method comprising the systemic, non-intrathecal administration of an omega conotoxin concurrently, separately or sequentially with a compound which inhibits neuronal excitation in amounts effective to induce an analgesic response.

In a further aspect, the present invention provides a method for inducing an analgesic response in a subject suffering from pain, the method comprising the systemic, non-intrathecal administration of an omega conotoxin concurrently, separately or sequentially with a compound which inhibits neuronal excitation in amounts effective to induce an analgesic response whilst not causing overt sedation.

Compounds which inhibit neuronal excitation function by reducing, decreasing or blocking pain signals being transmitted to the brain. The term “inhibits” includes “decreases”. Herein, these compounds are referred to herein as inter alia “neuronal excitation blockers”, “excitation blockers”, “neuronal excitation inhibitor” and “antagonists of neuronal excitation”. Such compounds include, without being limited to flupirtine or a pharmaceutically acceptable salt, derivative, homolog or analog thereof; retigabine or a pharmaceutically acceptable salt, derivative, homolog or analog thereof; compounds that cause opening of neuronal potassium channels; sodium channel blockers; a modulator of CB2 receptors; a modulator of TRPV1 receptors; a local anaesthetic; opioids; neurosteroids; alpha 2 adrenoceptor antagonists; NSAIDS; NMDA antagonists and calcium channel antagonists. In one embodiment, the omega conotoxin and the neuronal excitation inhibitor are administered in amounts effective to reduce the symptoms of cancer pain or inflammatory pain. Such an effective amounts include synergistic effective amounts. In addition, a subject may also be specifically selected on the basis of the type of pain and hence a selection step for a particular patient or subject also forms an aspect of the present invention.

In one aspect, the neuronal excitation inhibitor is an opioid, such as but not limited to fentanyl, oxycodone, codeine, dihydrocodeine, dihydrocodeinone enol acetate, morphine, desomorphine, apomorphine, diamorphine, pethidine, methadone, dextropropoxyphene, pentazocine, dextromoramide, oxymorphone, hydromorphone, dihydromorphine, noscapine, papverine, papvereturn, alfentanil, buprenorphine and tramadol and pharmaceutically acceptable salts, derivatives, homologs or analogs thereof as well as opioid agonists.

Yet another aspect relates to the use of an omega conotoxin either alone or in combination with flupirtine or retigabine or a pharmaceutically acceptable salt, derivative, homolog or analog thereof in the manufacture of a medicament for inducing an analgesic response in the treatment of pain, wherein the medicament is formulated for systemic, non-intrathecal administration.

Still another aspect is directed to the use of an omega conotoxin either alone or in combination with flupirtine or retigabine or a pharmaceutically acceptable salt, derivative, homolog or analog thereof in the manufacture of a medicament for inducing an analgesic response in the treatment of pain without inducing overt sedation, wherein the medicament is formulated for systemic, non-intrathecal administration.

A further aspect relates to the use of an omega conotoxin either alone or in combination with a neuronal excitation inhibitor, such as flupirtine or retigabine or a pharmaceutically acceptable salt, derivative, homolog or analog thereof, in the manufacture of one or more separate or combined medicaments for inducing an analgesic response to pain. In an aspect, the analgesia is induced without overt sedation. In an embodiment the omega conotoxin is CVID and is combined with a neuronal excitation inhibitor such as flupirtine or retigabine.

Even yet another aspect is directed to the use of an omega conotoxin and one or more sodium channel blockers in the manufacture of a medicament for inducing analgesia in response to cancer pain or inflammatory pain. Still yet another aspect provides for the use of an omega conotoxin and one or more sodium channel blockers in the manufacture of a medicament for inducing analgesia in response to cancer pain or inflammatory pain without overt sedation. Sodium channel blockers include without being limited to lamotrogine and mexiletine or a pharmaceutically acceptable salt, derivative, homolog or analog thereof.

In addition, the omega conotoxin may be used in combination with one or more local anesthetics such as but not limited to lignocaine, bupivacaine, ropivacaine, and procaine tetracaine or a pharmaceutically acceptable salt, derivative, homolog or analog thereof.

Furthermore, the omega conotoxin may be used in combination with one or more modulators of TRPV1 receptors, such as but not limited to capsaicin, capsazepine, Nb-VNA, Nv-VNA, SB-705498 and anadamide or a pharmaceutically acceptable salt, derivative, homolog or analog thereof.

Still further, the omega conotoxin may be used in combination with one or more modulators of CB2 receptors such as but not limited to SR144528, AM630 and anadamide or a pharmaceutically acceptable salt, derivative, homolog or analog thereof.

Reference to a “neuronal excitation inhibitor” also includes a sodium channel blocker, a local anaesthetic, a modulator of TRPV1 receptor and/or modulator of CB2 receptor. Equally, a sodium channel blocker, a local anaesthetic, a modulator of TRPV1 receptor and/or modulator of CB2 receptor may also be a neuronal excitation inhibitor.

A delivery system is also provided for inducing analgesia in response to pain in a subject comprising an omega conotoxin and a compound which decreases or inhibits neuronal excitation or a pharmaceutically acceptable salt, derivative, homolog or analog thereof. In one aspect the omega conotoxin is selected from CVID, GVIA, MVIIA and SNX-111. The delivery system may, for example, be in the form of a cream or an injection. The “injection” includes slow or controlled release injectables. The delivery system may also be a sustained release or slow release formulation, or a tamper proof formulation, or a pharmaceutical formulation or coated onto a stent, catheter or other mechanical device designed for use in a medical procedure.

The compounds according to the present invention may be administered, inter alia, orally, transmucosally, rectally including via suppository, subcutaneously, intravenously, intramuscularly, intraperitoneally, intragastrically, intranasally, transdermally, transmucosally, including rectal, buccal (sublingual), transnasal administration or intestinally or injected into a joint. As used herein, the phrase “systemic, non-intrathecal administration” specifically excludes the intrathecal administration of an omega conotoxin either alone or in combination with a neuronal excitation inhibitor. Hence, the present invention extends to the systemic administration of the medicament with the proviso that the systemic administration is not intrathecal administration. The present invention further contemplates nanoparticulate formulations which include nanocapsules, nanoparticles, microparticles, liposomes, nanospheres, microspheres, lipid particles, and the like. Such formulations increase the delivery efficacy and bioavailability and reduce the time for analgesic effect of the pain management agents. Nanoparticles generally comprise forms of the agents entrapped within a polymeric framework or other suitable matrix. Nanoparticle formulations are particularly useful for sparingly water soluble drugs. Such formulations also increase bioavailability. One method of formulation is a wet bead milling coupled to a spray granulation.

Methods and compositions are provided herein for use in treating pain. Generally this occurs without causing overt sedation. As used herein, “without causing overt sedation” includes inducing an analgesic effect without causing significant cognitive or general impairment of nervous system function (such as attention or wakefulness). Such effects on cognition leads to a change in the measurement that leads to an erroneous conclusion about the drug combination causing analgesia.

In addition, it is also contemplated that an embodiment, systemic, non-intrathecal administration of an omega conotoxin either alone or in combination with a neuronal excitation inhibitor induces an analgesic response to pain without causing one or more dose-limiting side-effects. Dose-limiting side-effects include orthostatic hypotension, sinus bradycardia, neurocardiogenic syncope and hypotension.

In one aspect, the omega conotoxin is combined with flupirtine or retigabine or pharmaceutically acceptable salt, derivative, homolog or analog thereof. The flupirtine or retigabine is administered at a dose of between about 50 μg to 2,000/mg, at intervals of between about 1 hour and about 50 hours and may be administered prior to, simultaneously with or following the omega conotoxin. These amounts can also be represented in terms of kg of body weight. Hence, the flupirtine or retigabine may be administered from 0.5 μg/kg body weight to 20 mg/kg body weight.

In a particular embodiment, the subject is a mammal, and in a most particular embodiment, the subject is a human. The subject or a group of subjects may be selected on the basis of the type of pain experienced. The “type” of pain may also be subjectively determined based on symptoms described by the subject. Hence, a therapeutic protocol is contemplated which comprises selecting a subject on the basis of symptoms of pain and administering to the subject an omega conotoxin and a neuronal excitation inhibitor wherein the treatment does not cause overt sedation.

A further aspect provides a system for the controlled release of an active compound selected from an omega conotoxin and a neuronal excitation inhibitor, wherein the system comprises:

(a) a deposit-core comprising an effective amount of a first active compound and having defined geometric form, and

(b) a support-platform applied to the deposit-core, wherein the support-platform contains a second active compound, and at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

As used herein, the first active compound is one of (i) an omega conotoxin or (ii) one or more neuronal excitation inhibitors. The second active compound may be (i) or (ii) above.

In another aspect, a system is described for the controlled release for an omega conotoxin and a neuronal excitation inhibitor where the system comprises:

(a) a deposit-core comprising an effective amount of the omega conotoxin and the neuronal excitation inhibitor; and

(b) a support platform applied to the deposit-core, the support platform comprising at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

Pain management protocols including point of care therapeutic protocols for controlling pain or the sensation of pain are also provided herein. The protocols include assessing a subject for pain type or causation of pain and systemically, non-intrathecally providing to the subject an omega conotoxin alone or in combination with a neuronal excitation inhibitor. The pain may be nociceptive pain or neuropathic pain such as inflammatory or cancer pain.

### BRIEF DESCRIPTION OF THE FIGURES

FIG. 1 is a graphical representation of the effect on pain of CVID and MVIIA administered either separately or in combination with a neuronal excitation inhibitor (flupirtine).

FIG. 2 is a graphical representation of a dose response curves for reversal of hyperalgesia in diabetic rates.

FIG. 3 is a graphical representation of a surface plot of reversal of STZ-induced hyperalgesia by leconotide and flupirtine alone and in combination.

FIG. 4 is a graphical representation of a comparison of maximum non-sedating doses and combinations o leconotide and ziconotide with flupirtine in reversal of hyperalgesia caused by Streptozotocin-induced diabetic neuropathy.

FIG. 5 is a graphical representation of a linear regression log dose response curves for flupirtine and morphine antinociception in a rat model of bone cancer.

FIG. 6 is a graphical representation of morphine dose versus dose of flupirtine.

FIG. 7 is a graphical representation of a dose response curve—leconotide alone in reversal of paw hyperalgesic caused by intratibial prostate cancer.

FIG. 8 is a graphical representation of morphine dose-response curves in cancer induced bone pain in rats.

### DETAILED DESCRIPTION

The singular forms “a”, “an” and “the” include plural aspects unless the context clearly dictates otherwise. Thus, for example, reference to “a neuronal excitation inhibitor” includes a single neuronal excitation inhibitor, as well as two or more neuronal excitation inhibitors; reference to “an omega conotoxin” includes a single omega conotoxin, as well as two or more omega conotoxins; reference to “the invention” includes one aspect or multiple aspects of an invention.

Terms such as “effective amount”, “amounts effective to”, “therapeutically effective amount” and “an analgesic effective amount” of an agent as used herein mean a sufficient amount of the agent (e.g. an omega conotoxin and/or flupirtine or retigabine) to provide the desired therapeutic or physiological effect or outcome, which includes achievement of pain reduction such as a sense of analgesia. Undesirable effects, e.g. side effects (such as overt sedation), are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation or the experience of the clinician. In particular, the methods and compositions described herein including the therapeutic protocol achieve analgesia of pain. In an embodiment, analgesia is achieved without overt sedation. Hence, the agent(s) is/are administered in amounts effective to induce analgesia whilst not causing overt sedation.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, emide, prodrug or derivative of a compound is a salt, ester, amide, prodrug or derivative that this not biologically or otherwise undesirable.

The terms “treating” and “treatment” as used herein refer to reduction in severity and/or frequency of pain associated with a condition being treated, elimination of symptoms and/or underlying cause of the pain, prevention of the occurrence of pain associated with a condition and/or its underlying cause and improvement or remediation or amelioration of pain following a condition. Hence, the treatment proposed herein reduces pain but this may be independent of the condition being treated.

“Treating” a subject may involve both treating the condition and reducing pain.

A “subject” as used herein refers to an animal, including a mammal such as a human who can benefit from the pharmaceutical formulations and methods of the present invention. There is no limitation on the type of animal that could benefit from the presently described pharmaceutical formulations and methods. A subject regardless of whether a human or non-human animal may be referred to as a subject, individual, patient, animal, host or recipient. The compounds and methods described herein have applications in human medicine, veterinary medicine as well as in general, domestic or wild animal husbandry.

The term “mammal” includes humans and non-human primates such as orangutangs, gorillas and marmosets as well as livestock animals, laboratory test animals, companion animals and captive wild animals. The subject may also be an avian species.

Examples of laboratory test animals include mice, rats, rabbits, simian animals, guinea pigs and hamsters. Rabbits, rodent and simian animals provide a convenient test system or animal model. Livestock animals include sheep, cows, pigs, goats, horses and donkeys.

In one aspect, a method is provided for inducing an analgesic response to pain in a subject. In this context the term “subject” is intended to include and encompass both humans and non-human animals. This aspect also includes, in one embodiment, the step of selecting a subject having pain to be a recipient of treatment. The selection process includes an assessment of symptoms of pain or symptoms of a condition likely to result in pain.

The term “pain” is intended to describe the subset of acute and chronic pain that results from nociceptive pain or neuropathic pain. Pain from cancer and inflammatory conditions is also contemplated.

Nociceptive pain is caused by activation of nociceptors and includes pain caused by cuts, bruises, bone fractures, crush injuries, burns, or tissue trauma.

Throughout this specification, the term “neuropathic pain” is to be understood to mean pain initiated or caused by a primary lesion or dysfunction within the nervous system. Examples of categories of neuropathic pain that may be treated by the methods of the present invention include monoradiculopathies, trigeminal neuralgia, postherpetic neuralgia, phantom limb pain, complex regional pain syndromes, back pain, neuropathic pain associated with AIDS and infection with the human immunodeficiency virus and the various peripheral neuropathies, including, but not limited to drug-induced and diabetic neuropathies.

In a further embodiment, the present invention extends to treating pain associated with any one or more of the following diseases which cause neuropathic pain or which have a neuropathic pain component: abdominal wall defect, abdominal migraine, achondrogenesis, achondrogenesis Type IV, achondrogenesis Type III, achondroplasia, achondroplasia tarda, achondroplastic dwarfism, Acquired humanimmunodeficiency Syndrome (AIDS), acute intermittent porphyria, acute porphyrias, acute shoulder neuritis, acute toxic epidermolysis, adiposa dolorosa, adrenal neoplasm, adrenomyeloneuropathy, adult dermatomyositis, amyotrophic lateral sclerosis, amyotrophic lateral sclerosis-polyglucosan bodies, AN, AN1, AN2, anal rectal malformations, anal stenosis, arachnitis, arachnoiditis ossificans, arachnoiditis, arteritis giant cell, arthritis, arthritis urethritica, ascending paralysis, astrocytoma grade I (Benign), astrocytoma grade II (Benign), athetoid cerebral palsy, barrett esophagus, barrett ulcer, benign tumors of the central nervous system, bone tumor-epidermoid cyst-polyposis, brachial neuritis, brachial neuritis syndrome, brachial plexus neuritis, brachial-plexus-neuropathy, brachiocephalic ischemia, brain tumors, brain tumors benign, brain tumors malignant, brittle bone disease, bullosa hereditaria, bullous cie, bullous congenital ichthyosiform erythroderma, bullous ichthyosis, bullous pemphigoid, Burkitt's lymphoma, Burkitt's lymphoma African type, Burkitt's lymphoma non-African type, calcaneal valgus, calcaneovalgus, cavernous lymphangioma, cavernous malformations, central form neurofibromatosis, cervical spinal stenosis, cervical vertebral fusion, Charcot's disease, Charcot-Marie-Tooth disease, Charcot-Marie-Tooth disease variant, Charcot-Marie-Tooth-Roussy-Levy disease, childhood dermatomyositis, chondrodysplasia punctata, chondrodystrophia calcificans congenita, chondrodystrophia fetalis, chondrodystrophic myotonia, chondrodystrophy, chondrodystrophy with clubfeet, chondrodystrophy epiphyseal, chondrodystrophy hyperplastic form, chondroectodermal dysplasias, chondrogenesis imperfecta, chondrohystrophia, chondroosteodystrophy, chronic adhesive arachnoiditis, chronic idiopathic polyneuritis (CIP), chronic inflammatory demyelinating polyneuropathy, chronic inflammatory demyelinating polyradiculoneuropathy, cicatricial pemphigoid, complex regional pain syndrome, congenital cervical synostosis, congenital dysmyelinating neuropathy, congenital hypomyelinating polyneuropathy, congenital hypomyelination neuropathy, congenital hypomyelination, congenital hypomyelination (onion bulb) polyneuropathy, congenital ichthyosiform erythroderma, congenital tethered cervical spinal cord syndrome, cranial arteritis, Crohn's disease, cutaneous porphyrias, degenerative lumbar spinal stenosis, demyelinating disease, diabetes mellitus diabetes insulin dependent, diabetes mellitus, diabetes mellitus addison's disease myxedema, discoid lupus, discoid lupus erythematosus, disseminated lupus erythematosus, disseminated neurodermatitis, disseminated sclerosis, eds kyphoscoliotic, eds kyphoscoliosis, eds mitis type, eds ocular-scoliotic, elastosis dystrophica syndrome, encephalofacial angiomatosis, encephalotrigeminal angiomatosis, enchondromatosis with multiple cavernous hemangiomas, endemic polyneuritis, endometriosis, eosinophilic fasciitis, epidermolysis bullosa, epidermolysis bullosa acquisita, epidermolysis bullosa hereditaria, epidermolysis bullosa letalias, epidermolysis hereditaria tarda, epidermolytic hyperkeratosis, epidermolytic hyperkeratosis, familial lumbar stenosis, familial lymphedema praecox, fibromyalgia, fibromyalgia-fibromyositis, fibromyositis, fibrositis, fibrous ankylosis of multiple joints, fibrous dysplasia, fragile x syndrome, generalized fibromatosis, guillain-barre syndrome, hemangiomatosis chondrodystrophica, hereditary sensory and autonomic neuropathy Type I, hereditary sensory and autonomic neuropathy Type II, hereditary sensory and autonomic neuropathy Type III, hereditary sensory motor neuropathy, hereditary sensory neuropathy type i, hereditary sensory neuropathy type i, hereditary sensory neuropathy type ii, hereditary sensory neuropathy type m, hereditary sensory radicular neuropathy type i, hereditary sensory radicular neuropathy type i, hereditary sensory radicular neuropathy type ii, herpes zoster, hodgkin disease, Hodgkin's disease, Hodgkin's lymphoma, hyperplastic epidermolysis bullosa, hypertrophic interstitial neuropathy, hypertrophic interstitial neuritis, hypertrophic interstitial radiculoneuropathy, hypertrophic neuropathy of refsum, idiopathic brachial plexus neuropathy, idiopathic cervical dystonia, juvenile (childhood) dermatomyositis (jdms), juvenile diabetes, juvenile rheumatoid arthritis, pes planus, leg ulcer, lumbar canal stenosis, lumbar spinal stenosis, lumbosacral spinal stenosis, lupus, lupus, lupus erythematosus, lymphangiomas, mononeuritis multiplex, mononeuritis peripheral, mononeuropathy peripheral, monostotic fibrous dysplasia, multiple cartilaginous enchondroses, multiple cartilaginous exostoses, multiple enchondromatosis, multiple myeloma, multiple neuritis of the shoulder girdle, multiple osteochondromatosis, multiple peripheral neuritis, multiple sclerosis, musculoskeletal pain syndrome, neuropathic amyloidosis, neuropathic beriberi, neuropathy of brachialpelxus syndrome, neuropathy hereditary sensory type i, neuropathy hereditary sensory type ii, nieman pick disease type a (acute neuronopathic form), nieman pick disease type b, nieman pick disease type c (chronic neuronopathic form), non-scarring epidermolysis bullosa, ochronotic arthritis, ocular herpes, onion-bulb neuropathy, osteogenesis imperfect, osteogenesis imperfecta, osteogenesis imperfecta congenita, osteogenesis imperfecta tarda, peripheral neuritis, peripheral neuropathy, perthes disease, polyarteritis nodosa, polymyalgia rheumatica, polymyositis and dermatomyositis, polyneuritis peripheral, polyneuropathy peripheral, polyneuropathy and polyradiculoneuropathy, polyostotic fibrous dysplasia, polyostotic sclerosing histiocytosis, postmyelographic arachnoiditis, primary progressive multiple sclerosis, psoriasis, radial nerve palsy, radicular neuropathy sensory, radicular neuropathy sensory recessive, reflex sympathetic dystrophy syndrome, relapsing-remitting multiple sclerosis, sensory neuropathy hereditary type i, sensory neuropathy hereditary type ii, sensory neuropathy hereditary type i, sensory radicular neuropathy, sensory radicular neuropathy recessive, sickle cell anemia, sickle cell disease, sickle cell-hemoglobin c disease, sickle cell-hemoglobin d disease, sickle cell-thalassemia disease, sickle cell trait, spina bifida, spina bifida aperta, spinal arachnoiditis, spinal arteriovenous malformation, spinal ossifying arachnoiditis, spinal stenosis, stenosis of the lumbar vertebral canal, still's disease, syringomyelia, systemic sclerosis, talipes calcaneus, talipes equinovarus, talipes equinus, talipes varus, talipes valgus, tandem spinal stenosis, temporal arteritis/giant cell arteritis, temporal arteritis, tethered spinal cord syndrome, tethered cord malformation sequence, tethered cord syndrome, tethered cervical spinal cord syndrome, thalamic pain syndrome, thalamic hyperesthetic anesthesia, trigeminal neuralgia, variegate porphyria, vertebral ankylosing hyperostosis amongst others.

In another embodiment, the present invention contemplates the use of compositions and methods comprising an omega conotoxin either alone or in combination with a neuronal excitation inhibitor in the treatment of pain associated with inflammatory conditions. The term “inflammatory pain” or a pain associated with inflammation is intended to describe the subset of acute and chronic pain that results from inflammatory processes, such as may arise in the case of infections, arthritis and neoplasia or tumor related hypertrophy. Inflammatory pain includes pain associated with rheumatoid arthritis, osteo-arthritis, psoriatic arthropathy, arthritis associated with other inflammatory and autoimmune conditions, degenerative conditions such as back strain and mechanical back pain or disc disease, post operative pain, pain from an injury such as a soft tissue bruise or strained ligament or broken bone, abscess or cellulitis, fibrositis or myositis.

Other examples of inflammatory conditions include, but are not limited to, inflammatory diseases and disorders which result in a response of redness, swelling, pain, and a feeling of heat in certain areas that is meant to protect tissues affected by injury or disease. Inflammatory diseases which include a pain component which can be relieved using the compositions and methods of the present invention include, without being limited to, acne, angina, arthritis, aspiration pneumonia, disease, empyema, gastroenteritis, inflammation, intestinal flu, NEC, necrotizing enterocolitis, pelvic inflammatory disease, pharyngitis, PID, pleurisy, raw throat, redness, rubor, sore throat, stomach flu and urinary tract infections, chronic inflammatory demyelinating polyneuropathy, chronic inflammatory demyelinating Polyradiculoneuropathy, chronic inflammatory demyelinating polyneuropathy, chronic inflammatory demyelinating polyradiculoneuropathy.

In a further embodiment, the present invention provides methods and compositions for alleviating the pain associated with cancer (“cancer pains”).

In one particular embodiment, an omega conotoxin, either alone or in combination with a neuronal excitation inhibitor is used during or following cancer treatment. Examples of cancers which contain a pain component that may be relieved using the compositions and methods of the present invention include but are not limited to abll protooncogene, aids related cancers, acoustic neuroma, acute lymphocytic leukaemia, acute myeloid leukaemia, adenocystic carcinoma, adrenocortical cancer, agnogenic myeloid metaplasia, alopecia, alveolar soft-part sarcoma, anal cancer, angiosarcoma, aplastic anaemia, astrocytoma, ataxia-telangiectasia, basal cell carcinoma (skin), bladder cancer, bone cancers, bowel cancer, brain stem glioma, brain and cns tumors, breast cancer, cns tumors, carcinoid tumors, cervical cancer, childhood brain tumors, childhood cancer, childhood leukaemia, childhood soft tissue sarcoma, chondrosarcoma, choriocarcinoma, chronic lymphocytic leukaemia, chronic myeloid leukaemia, colorectal cancers, cutaneous t-cell lymphoma, dermatofibrosarcoma-protuberans, desmoplastic-small-round-cell-tumor, ductal carcinoma, endocrine cancers, endometrial cancer, ependymoma, esophageal cancer, ewing's sarcoma, extra-hepatic bile duct cancer, eye cancer, eye: melanoma, retinoblastoma, fallopian tube cancer, fanconi anaemia, fibrosarcoma, gall bladder cancer, gastric cancer, gastrointestinal cancers, gastrointestinal-carcinoid-tumor, genitourinary cancers, germ cell tumors, gestational-trophoblastic-disease, glioma, gynaecological cancers, haematological malignancies, hairy cell leukaemia, head and neck cancer, hepatocellular cancer, hereditary breast cancer, histiocytosis, hodgkin's disease, human papillomavirus, hydatidiform mole, hypercalcemia, hypopharynx cancer, intraocular melanoma, islet cell cancer, kaposi's sarcoma, kidney cancer, langerhan's-cell-histiocytosis, laryngeal cancer, leiomyosarcoma, leukaemia, lifraumeni syndrome, lip cancer, liposarcoma, liver cancer, lung cancer, lymphedema, lymphoma, hodgkin's lymphoma, non-hodgkin's lymphoma, male breast cancer, malignant-rhabdoid-tumor-of-kidney, medulloblastoma, melanoma, merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, nijmegen breakage syndrome, non-melanoma skin cancer, non-small-cell-lung-cancer-(nsclc), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumors, pituitary cancer, polycythemia vera, prostate cancer, rare-cancers-and-associated-disorders, renal cell carcinoma, retinoblastoma, rhabdomyosarcoma, rothmund-thomson syndrome, salivary gland cancer, sarcoma, schwannoma, sezary syndrome, skin cancer, small cell lung cancer (sclc), small intestine cancer, soft tissue sarcoma, spinal cord tumors, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis-/-ureter), trophoblastic cancer, urethral cancer, urinary system cancer, uroplakins, uterine sarcoma, uterus cancer, vaginal cancer, vulva cancer, waldenstrom's-macroglobulinemia or Wilms' tumor.

In an embodiment, an analgesic response is induced without inducing overt sedation to pain being suffered by a subject, including a human subject. In this context, the terms “analgesia” and “analgesic response” are intended to describe a state of reduced sensibility to pain, which occurs without overt sedation and in an embodiment without an effect upon the sense of touch In another aspect, the sensibility to pain is completely, or substantially completely, removed. To assess the level of reduction of sensibility to pain associated with the analgesia induced by the methods according to the present invention it is possible to conduct tests such as the short form McGill pain questionnaire and/or visual analog scales for pain intensity and/or verbal rating scales for pain intensity and/or measurement of tactile allodynia using von Frey hairs or similar device. These tests are standard tests within the art and would be well known to the skilled person. Hence, a reduction to the sensibility to pain can be represented subjectively or qualitatively as a percentage reduction by at least 10%, at least 20%, at least 50%, at least 70% or at least 85% including at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 or 85%.

Accordingly, a method is contemplated for inducing an analgesic response to pain in a subject, the method comprising the systemic, non-intrathecal administration to the subject of an amount of an omega conotoxin, or a pharmaceutically acceptable salt, derivative, homolog or analog thereof, effective to reduce the level of or otherwise ameliorate the sensation of pain. In another embodiment, a method is provided for inducing an analgesic response to pain, the method comprising the systemic, non-intrathecal administration to the subject of an amount of an omega conotoxin, or a pharmaceutically acceptable salt, derivative, homolog or analog thereof, effective to reduce the level of or otherwise ameliorate the sensation of pain whilst not causing overt sedation. Examples of particular omega contoxins contemplated for use are CVID (also known as AM336 and leconotide), GVIA, MVIIA (also known as Ziconotide and Prialt) and SNX-111. Omega conotoxins contemplated for use in the methods or compositions of the present invention are also described in PCT Application No. PCT/AU99/00288, the contents of which are incorporated herein by reference.

In a related aspect, the present invention provides a method for inducing an analgesic response to pain in a subject, the method comprising the systemic, non-intrathecal administration to the subject an amount of an omega conotoxin in combination with a neuronal excitation inhibitor effective to reduce the level of, or otherwise ameliorate, the sensation of pain.

In another aspect, the present invention provides a method for inducing an analgesic response to pain in a subject, the method comprising the systemic, non-intrathecal administration to the subject an amount of an omega conotoxin in combination with a neuronal excitation inhibitor effective to reduce the level of or otherwise ameliorate the sensation of pain whilst not causing overt sedation.

Another aspect provides a method of inducing analgesia in a subject suffering pain by administering to the subject an omega conotoxin concurrently, separately or sequentially with respect to a neuronal excitation inhibitor, such as flupirtine or retigabine, or a pharmaceutically acceptable salt, derivative, homolog or analog thereof, in an amount effective to reduce the level of or otherwise ameliorate the sensation of pain associated with cancer or inflammation without inducing overt sedation.

In a further aspect provides a method of inducing analgesia without overt sedation in a subject suffering pain by administering to the subject an omega conotoxin concurrently, separately or sequentially with respect to a neuronal excitation inhibitor, such as flupirtine or retigabine, or a pharmaceutically acceptable salt, derivative, homolog or analog thereof, in an amount effective to reduce the level of or otherwise ameliorate the sensation of pain associated with cancer or inflammation without inducing overt sedation.

Still another aspect contemplates combination therapy in the treatment of pain wherein the treatment of the disease, condition or pathology is conducted in association with pain management using an omega conotoxin, such as CVID or MVIIA, and a neuronal excitation inhibitor, such as flupirtine or retigabine or a pharmaceutically acceptable salt, derivative, homolog or analog thereof and optionally in addition to an analgesic agent.

In yet another aspect contemplates combination therapy in the treatment of pain without inducing overt sedation wherein the treatment of the disease, condition or pathology is conducted in association with pain management using an omega conotoxin, such as CVID or MVIIA, and a neuronal excitation inhibitor, such as flupirtine or retigabine or a pharmaceutically acceptable salt, derivative, homolog or analog thereof and optionally in addition to an analgesic agent.

Even still another aspect provides a method for inducing an analgesic response to pain in a subject comprising systemic, non-intrathecal administration to the subject an amount of an omega conotoxin and a sodium channel blocker such as but not limited to lamotrogine and mexiletine or a pharmaceutically acceptable salt, derivative, homolog or analog thereof to reduce the level of or otherwise ameliorate the sensation of.

In a related aspect the present invention provides a method for inducing an analgesic response to pain without inducing overt sedation in a subject comprising systemic, non-intrathecal administration to the subject an amount of an omega conotoxin and a sodium channel blocker such as but not limited to lamotrogine and mexiletine or a pharmaceutically acceptable salt, derivative, homolog or analog thereof to reduce the level of or otherwise ameliorate the sensation of pain without inducing overt sedation.

Yet another aspect is directed to a method for inducing an analgesic response to pain in a subject comprising administering to the subject an amount of an omega conotoxin and a local anaesthetic such as lignocaine, bupivacaine, ropivacaine, and procaine tetracaine or a pharmaceutically acceptable salt, derivative, homolog or analog thereof to reduce the level of or otherwise ameliorate the sensation of pain.

In another aspect is directed to a method for inducing an analgesic response to pain without inducing overt sedation in a subject comprising administering to the subject an amount of an omega conotoxin and a local anaesthetic such as lignocaine, bupivacaine, ropivacaine, and procaine tetracaine or a pharmaceutically acceptable salt, derivative, homolog or analog thereof to reduce the level of or otherwise ameliorate the sensation of pain without inducing overt sedation,

The omega conotoxins may also be used in combination with one or more modulators of TRPV1 receptors, such as but not limited to capsaicin, capsazepine, Nb-VNA, Nv-VNA, SB-705498 and anadamide or a pharmaceutically acceptable salt, derivative, homolog or analog thereof.

Still further, the omega conotxins may be used in combination with one or more modulators of CB2 receptors such as but not limited to SR144528, AM630 and anadamide or a pharmaceutically acceptable salt, derivative, homolog or analog thereof.

By the term “overt sedation” it is intended to convey that the methods (and compositions) described herein do not result in a level of sedation of the patient or subject being treated which shows significant, visible or apparent drowsiness or unconsciousness of the patient being treated. Thus, the treatment methods and compositions herein, in one embodiment, do not result in sleepiness or drowsiness in the patient that interfere with, or inhibit, the activities associated with day to day living, such as driving a motor vehicle or operating machinery for human subjects, or feeding and grooming for animal subjects. The term “without overt sedation” also means inducing an analgesic effect without causing significant cognitive or general impairment of nervous system function (such as attentiveness or wakefulness). Such effects on cognition can lead to a change in the measurement that leads to an erroneous conclusion about the level or type of pain or effect of amelioration of symptoms.

The term “omega conotoxin” is intended to encompass known and as yet unknown compounds (including pharmaceutically acceptable salts, derivatives, homologs or analogs thereof) that are effective for treatment of pain in mammals, including compounds which act directly on N-type calcium channels. Omega conotoxins are reasonably small peptides (typically peptides of 24-32 amino acids in length) with six characteristic cysteine substitutions and a pattern of disulphide bonds. Examples of such omega conotoxins include CVID (also known as AM336 and leconotide), GVIA, MVIIA (also known as Ziconotide and Prialt) and SNX-111. Omega conotoxins contemplated for use in the methods or compositions of the present invention are also described in PCT Application No. PCT/AU99/00288, the contents of which are incorporated herein by reference.

As used herein, compounds which inhibit neuronal excitation include, without being limited to, flupirtine or retigabine; compounds which cause opening of neuronal potassium channels, opioids, neurosteroids, NSAIDS; NMDA receptor antagonists and calcium channel antagonists.

Reference to a “neuronal excitation inhibitor” also include a sodium channel blocker, a local anaesthetic, a modulator of TRPV1 receptor and/or modulator of CB2 receptor. Equally, a sodium channel blocker, a local anaesthetic, a modulator of TRPV1 receptor and/or modulator of CB2 receptor may also be a neuronal excitation inhibitor.

Potassium channels openers contemplated for use in the present invention include, without being limited to flupirtine, Retigabine, WAY-133537, ZD6169, Celikalim, NN414, arycyclopropylcarboxylic amides, 3-(pyridinyl-piperazin-1-YL)-phenylethyl amides, cromakalim, pinacidil, P1060, SDZ PC0400, minoxidil, nicrandil, BMS-204352, cromokalim, leveromakalim, lemakalim, diazoxide, charybdotoxin, glyburide and 4-aminopyridine.

Sodium channel blockers include lamotrogine and mexiletine.

Local anesthetics include lignocaine, bupivacaine, ropivacaine, procaine and tetracaine.

A modulator of TRPV1 receptor includes but is not limited to capsaicin, capsazepine, Nb-VNA, Nv-VNA, SB-705498 and anadamide or a pharmaceutically acceptable salt, derivative, homolog or analog thereof.

The modulator may be an agonist or an antagonist of the TRPV1 receptor. SB-705498 is an example of an antagonist and capsaicin, capsazepine, Nb-VNA, Nv-VNA and anadamide are examples of agonists.

A modulator of CB2 receptor includes but is not limited to SR144528, AM630 and anandamide or a pharmaceutically acceptable salt, derivative, homolog or analog thereof. The modulator may be an agonist or an antagonist of the CB2 receptor.

As used herein, opioid compounds (opioids) include any compound that is physiologically acceptable in animal systems and is a full or at least partial agonist of an opioid receptor, Opioid compounds are well known and include naturally occurring compounds derived from opium such as codeine, morphine and papavarine as well as derivatives of such compounds that generally have structural similarity as well as other structurally unrelated compounds that agonise an opioid receptor present in a mammalian system. Specific examples of opioid compounds contemplated by the present invention include: fentanyl, oxycodone, codeine, dihydrocodeine, dihydrocodeinone enol acetate, morphine, desomorphine, apomorphine, diamorphine, pethidine, methadone, dextropropoxyphene, pentazocine, dextromoramide, oxymorphone, hydromorphone, dihydromorphine, noscapine, nalbuphine papaverine, papavereturn, alfentanil, buprenorphine and tramadol and pharmaceutically acceptable salts, derivatives, homologs or analogs thereof.

Neurosteroids contemplated for use in the present invention include alphadolone and other pregnanediones and salts and derivates thereof (e.g. alphadolone mono and bi glucuronides) and other neurosteroids that cause antinociception without overt sedation by interaction with spinal cord GABAa receptors.

As used herein, an NMDA receptor antagonist is an agent which blocks or inhibits the activity and/or function of NMDA receptors. Hence, the present invention extends to functional NMDA antagonists as well as structural NMDA antagonists. The NMDA receptor is a cell-surface protein complex, widely distributed in the mammalian central nervous system that belongs to the class of ionotropic-glutamate receptors. It is involved in excitatory-synaptic transmission and the regulation of neuronal growth. The structure comprises a ligand-gated/voltage-sensitive ion channel. The NMDA receptor is highly complex and is believed to contain at least five distinct binding (activation) sites: a glycine-binding site, a glutamate-binding site (NMDA-binding site); a PCP-binding site, a polyamine-binding site, and a zinc-binding site. In general, a receptor antagonist is a molecule that blocks or reduces the ability of an agonist to activate the receptor. As used herein, an “NMDA-receptor antagonist” means any compound or composition, known or to be discovered, that when contacted with an NMDA receptor in vivo or in vitro, inhibits the flow of ions through the NMDA-receptor ion channel. A “functional” NMDA antagonist includes agents which raise the threshold for NMDA receptor activation Activating NMDA receptors increases cell excitability. Any drug that inhibits or decreases neuronal excitation in the CNS can potentially be a “functional” NMDA receptor antagonist because it decreases the excitation caused by NMDA receptor agonists. All such agents may be used in combination with an omega conotoxin to achieve a desired analgesic effect.

An NMDA-receptor antagonist can contain one or more chiral centers and/or double bonds and, therefore, exist as stereoisomers, such as double-bond isomers (i.e., geometric isomers), enantiomers, or diastereomers. As used herein, the term “NMDA-receptor antagonist” encompass all such enantiomers and stereoisomers, that is, both the stereomerically-pure form (e.g., geometrically pure, enantiomerically pure, or diastereomerically pure) and enantiomeric and stereoisomeric mixtures, e.g., racemates. The term “NMDA-receptor antagonist” further encompasses all pharmaceutically acceptable salts, all complexes (e.g., hydrates, solvates, and clathrates), and all prodrugs of NMDA-receptor antagonist.

NMDA-receptor antagonists suitable for use in the present invention can be identified by testing NMDA-receptor antagonists for antinociceptive properties according to standard pain models. See e.g., Sawynok et al. Pain 82:149, 1999; Sawynok et al. Pain 80:45, 1999.

In an aspect, the NMDA-receptor antagonist is a non-competitive NMDA-receptor antagonists, more particularly, ketamine, even more particularly, ketamine hydrochloride.

As used herein the meaning of the phrase “NMDA-receptor antagonist” encompasses any compound or composition that antagonizes the NMDA receptor by binding at the glycine site. For a review on glycine-site NMDA-receptor antagonists, see Leeson, P. D. Drug Design for Neuroscience 13:338-381, 1993. Glycine-site NMDA-receptor antagonists can be identified by standard in vitro and in vivo assays. See, for example, the assays described in U.S. Pat. No. 6,251,903); U.S. Pat. No. 6,191,165; Grimwood et al. Molecular Pharmacology 4:923 1992; Yoneda et al. J Neurochem 62:102, 1994; and Mayer et al. J Neurophysiol 645, 1988.

Glycine-site NMDA-receptor antagonists include, but are not limited to, glycinamide, threonine, D-serine, felbamate, 5,7-dichlorokynurenic acid, and 3-amino-1-hydroxy-2-pyrrolidone (HA-966), diethylenetriamine, 1,10-diaminodecane, 1,12-diaminododecane, and ifenprodil and those described in U.S. Pat. Nos. 6,251,903; 5,914,403; U.S. Pat. No. 5,863,916; U.S. Pat. No. 5,783,700; and U.S. Pat. No. 5,708,168.

As used herein the meaning of the phrase “NMDA-receptor antagonist” encompasses any compound or composition that antagonizes the NMDA receptor by binding at the glutamate site also referred to herein as “competitive NMDA-receptor antagonists”; see, for example, Olney & Farber Neuropsychopharmacology 13:335, 1995.

Competitive NMDA antagonists include, but are not limited to, 3-((−)-2-carboxypiperazin-4-ylpropyl-1-phosphate (CPP); 3-(2-c arboxypiperzin-4-yl)-prpenyl-1-phosphonate (CPP-ene); 1-(cis-2-carboxypiperidine-4-yl)methyl-1-phosphonic acid (CGS 19755), D-2-Amino-5-phosphonopentanoic acid (AP5); 2-amino-phosphonoheptanoate (AP7); D,L-(E)-2-amino-4-methyl-5-phosphono-3-pentenoic acid carboxyethyl ester (CGP39551); 2-amino-4-methyl-5-phosphono-pent-3-enoic acid (CGP 40116); (4-phosphono-but-2-enylamino)-acetic acid (PD 132477); 2-amino-4-oxo-5-phosphono-pentanoic acid (MDL 100,453); 3-((phosphonylmethyl)-sulfinyl)-D,L-alanine; amino-(4-phosphonomethyl-phenyl)-acetic acid (PD 129635); 2-amino-3-(5-chloro-1-phosphonomethyl-1H-benzoimidazol-2-yl)-propionic acid; 2-amino-3-(3-phosphonomethyl-quinoxalin-2-yl)-propionic acid; 2-amino-3-(5-phosphonomethyl-biphenyl-3-yl)-propionic acid (SDZ EAB 515); 2-amino-3-[2-(2-phosphono-ethyl)-cyclohexyl]-propionic acid (NPC 17742); 4-(3-phosphono-propyl)-piperazine-2-carboxylic acid (D-CPP); 4-(3-phosphono-allyl)-piperazine-2-carboxylic acid (D-CPP-ene); 4-phosphonomethyl-piperidine-2-carboxylic acid (CGS 19755); 3-(2-phosphono-acetyl)-piperidine-2-carboxylic acid (MDL 100,925); 5-phosphono-1,2,3,4-tetrahydro-isoquinoline-3-carboxylic acid (SC 48981); 5-(2-phosphono-ethyl)-1,2,3,4-tetrahydro-isoquinoline-3-carboxylic acid (PD 145950); 6-phosphonomethyl-decahydro-isoquinoline-3-carboxylic acid (LY 274614); 4-(1H-tetrazol-5-ylmethyl)-piperidine-2-carboxylic acid (LY 233053 and 235723); and 6-(1H-Tetrazol-5-ylmethyl)-decahydro-isoquinoline-3-carboxylic acid (LY 233536).

As used herein the meaning of the phrase “NMDA-receptor antagonist” encompasses any compound or composition that antagonizes the NMDA receptor by binding at the PCP (phencyclidine) site, referred to herein as “non-competitive NMDA-receptor antagonists”.

Non-competitive NMDA-receptor antagonists can be identified using routine assays, for example, those described in U.S. Pat. No. 6,251,948 (issued Jun. 26, 2001); U.S. Pat. No. 5,985,586 (issued Nov. 16, 1999), and U.S. Pat. No. 6,025,369 (issued Feb. 15, 2000); Jacobson et al. J Pharmacol Exp Ther 110:243, 1987; and Thurkauf et al. J Med Chem 31:2257, 1988, all of which citations are hereby expressly incorporated herein by reference.

Examples of non-competitive NMDA-receptor antagonists that bind at the PCP site include, but are not limited to, ketamine, phencyclidine, dextromethorphan, dextrorphan, dexoxadrol, dizocilpine (MK-801), remacemide, thienylcyclohexylpiperidine (TCP), N-allylnometazocine (SKF 10,047), cyclazocine, etoxadrol, (1,2,3,4,9,9a-hexahydro-fluoren-4a-yl)-methyl-amine (PD 137889); (1,3,4,9,10,10a-hexahydro-2H-phenanthren-4a-yl)-methyl-amine (PD 138289); PD 138558, tiletamine, kynurenic acid, 7-chloro-kynurenic acid, and memantine; and quinoxalinediones, such as 6-cyano-7-nitroquinoxaline-2,3-dione (CNQX) and 6,7-dinitro-quinoxaline-2,3-dione (DNQX).

As used herein the meaning of “NMDA-receptor antagonist” encompasses compounds that block the NMDA receptor at the polyamine binding site, the zinc-binding site, and other NMDA-receptor antagonists that are either not classified herein according to a particular binding site or that block the NMDA receptor by another mechanism. Examples of NMDA-receptor antagonists that bind at the polyamine site include, but are not limited to, spermine, spermidine, putrescine, and arcaine. Examples of assays useful to identify NMDA-receptor antagonists that act at the zinc or polyamine binding site are disclosed in U.S. Pat. No. 5,834,465 (issued Nov. 10, 1998), hereby expressly incorporated by reference herein.

Other NMDA-receptor antagonists include, but are not limited to, amantadine, eliprodil, lamotrigine, riluzole, aptiganel, flupirtine, celfotel, levemopamil, 1-(4-hydroxy-phenyl)-2-(4-phenylsulfanyl-piperidin-1-yl)-propan-1-one; 2-[4-(4-fluoro-benzoyl)-piperidin-1-yl]-1-naphthalen-2-yl-ethanone (E 2001); 3-(1,1-dimethyl-heptyl)-9-hydroxymethyl-6,6-dimethyl-6a,7,8,10a-tetrahydro-6H-benzo[c]chromen-1-ol (HU-211); 1-{4-[1-(4-chloro-phenyl)-1-methyl-ethyl]-2-methoxy-phenyl}-1H-[1,2,4]triazole-3-carboxylic acid amide (CGP 31358); acetic acid 10-hydroxy-7,9,7′,9′-tetramethoxy-3,3′-dimethyl-3,4,3′,4′-tetrahydro-1H, 1′H-[5,5′]bi[benzo[g]isochromenyl]-4-yl ester (ES 242-1); 14-hydroxy-11-isopropyl-10-methyl-5-octyl-10,13-diaza-tricyclo[6.6.1.04,15]pentadeca-1,4,6,8(15)-tetraen-12-one; and 4,5-dioxo-4,5-dihydro-1H-benzo[g]indole-2,7,9-tricarboxylic acid (PQQ) and pharmaceutically acceptable salts thereof.

Calcium channel antagonists include diltiazem, ziconotide (MVIIA), CVID (AM336 (leconotide)), NMED-160, cilnidipine, GABApentin and pregabalin.

NSAIDS include, without being limited to, NSAIDS, such as acetaminophen (Tylenol, Datril, etc.), aspirin, ibuprofen (Motrin, Advil, Rufen, others), choline magnesium salicylate (Triasate), choline salicylate (Anthropan), diclofenac (voltaren, cataflam), diflunisal (dolobid), etodolac (iodine), fenoprofen calcium (nalfon), fluorobiprofen (ansaid), indomethacin (indocin, indometh, others), ketoprofen (orudis, oruvail), ketorolac tromethamine (toradol), magnesium salicylate (Doan's, magan, mobidin, others), meclofenamate sodium (meclomen), mefenamic acid (relafan), oxaprozin (daypro), piroxicam (feldene), sodium salicylate, sulindac (clinoril), tolmetin (tolectin), meloxicam, nabumetone, naproxen, lornoxicam, nimesulide, indoprofen, remifenzone, salsalate, tiaprofenic acid, flosulide, and the like.

The phrase “pharmaceutically acceptable salt, derivative, homologs or analogs” is intended to convey any pharmaceutically acceptable tautomer, salt, pro-drug, hydrate, solvate, metabolite or other compound which, upon administration to the subject, is capable of providing (directly or indirectly) the compound concerned or a physiologically (e.g. analgesically) active compound, metabolite or residue thereof. An example of a suitable derivative is an ester formed from reaction of an OH or SH group with a suitable carboxylic acid, for example C₁₋₃alkyl-CO₂H, and HO₂C—(CH₂)ₙ—CO₂H (where n is 1-10 such as 1, 2, 3, 4, 5, 6, 7, 8, 9, 10, but particularly 1-4), and CO₂H—CH₂-phenyl.

Thus, the active compounds may be in crystalline form, either as the free compounds or as solvates (e.g. hydrates). Methods of solvation are generally known within the art.

The salts of the active compounds of the invention are preferably pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulfuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulfphonic, toluenesulphonic, benzenesulphonic, salicyclic, sulphanilic, aspartic, glutamic, edetic, stearic, palmitic, oleic, lauric, pantothenic, tannic, ascorbic and valeric acids.

The term “pro-drug” is used herein in its broadest sense to include those compounds which can be converted in vivo to the compound of interest (e.g. by enzymatic or hydrolytic cleavage). Examples thereof include esters, such as acetates of hydroxy or thio groups, as well as phosphates and sulphonates. Processes for acylating hydroxy or thio groups are known in the art, e.g. by reacting an alcohol (hydroxy group), or thio group, with a carboxylic acid. Other examples of suitable pro-drugs are described in Bundgaard Design of Prodrugs, Elsevier 1985, the disclosure of which is included herein in its entirety by way of reference.

The term “metabolite” includes any compound into which the active agents can be converted in vivo once administered to the subject. Examples of such metabolites are glucuronides, sulphates and hydroxylates.

It will be understood that active agents as described herein may exist in tautomeric forms. The term “tautomer” is used herein in its broadest sense to include compounds capable of existing in a state of equilibrium between two isomeric forms. Such compounds may differ in the bond connecting two atoms or groups and the position of these atoms or groups in the compound. A specific example is keto-enol tautomerism.

The compounds of the present invention may be electrically neutral or may take the form of polycations, having associated anions for electrical neutrality. Suitable associated anions include sulfate, tartrate, citrate, chloride, nitrate, nitrite, phosphate, perchlorate, halosulfonate or trihalomethylsulfonate.

The active agents may be administered for therapy by any suitable route, other than intrathecally. It will be understood that the active agents are administered in one embodiment via a route that does not result in overt sedation of the subject, or result in dose-limiting side effects. Suitable routes of administration may include oral, rectal, nasal, inhalation of aerosols or particulates, topical (including buccal and sublingual), transdermal, vaginal, intravesical and parenteral (including subcutaneous, intramuscular, intravenous, intrasternal, intra-articular, injections into the joint, and intradermal). In one embodiment, administration of the active agent is by a route resulting in first presentation of the compound to the stomach of the subject. In this embodiment, the active agents are generally administered via an oral route. In another embodiment the active agents are administered by the transdermal route. However, it will be appreciated that the route may vary with the condition and age of the subject, the nature of the pain being treated, its location within the subject and the judgement of the physician or veterinarian. It will also be understood that individual active agents may be administered by the same or different distinct routes. The individual active agents may be administered separately or together directly into a joint involved with an inflammatory painful process.

As used herein, an “effective amount” refers to an amount of active agent that provides the desired analgesic activity when administered according to a suitable dosing regime. The amount of active agent is generally an amount that provides the desired analgesic activity. In one aspect, this occurs without causing overt sedation or dose limiting side-effects or drug tolerance. Dosing may occur at intervals of several minutes, hours, days, weeks or months. Suitable dosage amounts and regimes can be determined by the attending physician or veterinarian. For example, flupirtine or retigabine or pharmaceutically acceptable salts, derivatives, homologs or analogs thereof, may be administered in amounts o about 50 μg to about 2,000 mg including 100 μg, 200 μg, 300 μg, 500 μg, 800 μg, 1,000 μg, 10 mg, 20 mg, 50 mg, 100 mg, 500 mg, 1,000 mg, 1,500 mg and 2,000 mg or an amount in between. Alternatively, flupirtine or retigabine may be administered at a rate of between about 0.5 μg to about 20 mg/kg by body weight every from about 1 hour to up to about 50 hours, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 hours in amounts of 0.5 μg, 1 μg, 10 μg, 100 μg, 1 mg, 10 mg or 20 mg/kg body weight. Particularly useful times are from about 6 hours to about 24 hours, such as 6, 7, 8, 9, 10, 11, 12, 13, 14, 15, 16, 17, 18, 19, 20, 21, 22, 23, 24. Even more particular useful times are between from about 12 to about 24 hours. Such as 12, 13, 14, 15, 16, 17, 18, 19, 20, 21, 22, 23 or 24 hours. Dosing of the analgesic agent, such as an opioid, can be determined by the attending physician in accordance with dosing rates in practice. For example, fentanyl can be administered in an amount of about 100 μg whereas morphine may be administered in an amount of 10 mg, also on an hourly basis. The administration amounts may be varied if administration is conducted more or less frequently, such as by continuous infusion, by regular dose every few minutes (e.g. 1, 2, 3 or 4 minutes) or by administration every 5, 10, 20, 30 or 40 minutes (e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39 or 40 minutes) or every 1, 2, 3, 4, 5, 6, 7, 8, 9, 10, 11, 12, 13, 14, 15, 16, 17, 18, 19, 20, 21, 22, 23 or 24 hours or up to 50 hours such as, for example, 25, 26, 27, 28, 29, 30, 31, 32, 33, 34, 35, 36, 37, 38, 39, 40, 41, 42, 43, 44, 45, 46, 47, 48, 49 or 50 hours. In many instances, administration is conducted simply on the basis of when the patient requires pain relief.

Accordingly, a treatment protocol is contemplated for treating pain in a subject, the protocol comprising the steps of systemic, non-intrathecal administration to the subject an effective amount of an analgesic agent in conjunction with omega toxin and an inhibitor of neuronal excitation.

In another embodiment, a treatment protocol is provided for treating pain without inducing overt sedation in a subject, the protocol comprising the steps of systemic, non-intrathecal administration to the subject an effective amount of an analgesic agent in conjunction with omega toxin and an inhibitor of neuronal excitation.

A further aspect also provides a composition comprising an omega conotoxin with an inhibitor of neuronal excitation together with one or more pharmaceutically acceptable additives and optionally other medicaments. The pharmaceutically acceptable additives may be in the form of carriers, diluents, adjuvants and/or excipients and they include all conventional solvents, dispersion agents, fillers, solid carriers, coating agents, antifungal or antibacterial agents, dermal penetration agents, surfactants, isotonic and absorption agents and slow or controlled release matrices. The active agents may be presented in the form of a kit of components adapted for allowing concurrent, separate or sequential administration of the active agents. Each carrier, diluent, adjuvant and/or excipient must be “pharmaceutically acceptable” in the sense of being compatible with the other ingredients of the composition and physiologically tolerated by the subject. The compositions may conveniently be presented in unit dosage form and may be prepared by methods well known in the art of pharmacy. Such methods include the step of bringing into association the active ingredient with the carrier, which constitutes one or more accessory ingredients. In general, the compositions are prepared by uniformly and intimately bringing into association the active ingredient with liquid carriers, diluents, adjuvants and/or excipients or finely divided solid carriers or both, and then if necessary shaping the product.

Compositions of the present invention suitable for oral administration may be presented as discrete units such as capsules, sachets or tablets each containing a predetermined amount of the active ingredient; as a powder or granules; as a solution or a suspension in an aqueous phase or non-aqueous liquid; or as an oil-in-water liquid emulsion or a water-in-oil emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. inert diluent, preservative disintegrant, sodium starch glycollate, cross-linked povidone, cross-linked sodium carboxymethyl cellulose) surface-active or dispersing agent. Moulded tablets may be made my moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for parenteral administration include aqueous and non-aqueous isotonic sterile injection solutions which may contain anti-oxidants, buffers, bacteriostats and solutes which render the composition isotonic with the blood of the intended subject; and aqueous and non-aqueous sterile suspensions which may include suspended agents and thickening agents. The compositions may be presented in a unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described. When reconstituted these can be in the form of aqueous solution, dissolved in water, isotonic saline or a balanced salt solution. Additionally, when reconstituted the product could be a suspension in which the compound(s) is/are dispersed in the liquid medium by combination with liposomes or a lipid emulsion such as soya bean.

Compositions suitable for topical administration to the skin, i.e. transdermal administration, may comprise the active agents dissolved or suspended in any suitable carrier or base and may be in the form of lotions, gels, creams, pastes, ointments and the like. Suitable carriers may include mineral oil, propylene glycol, waxes, polyoxyethylene and long chain alcohols. Transdermal devices, such as patches may also be used and may comprise a microporous membrane made from suitable material such as cellulose nitrate/acetate, propylene and polycarbonates. The patches may also contain suitable skin adhesive and backing materials.

The active compounds described herein may also be presented as implants, which may comprise a drug bearing polymeric device wherein the polymer is biocompatible and non-toxic. Suitable polymers may include hydrogels, silicones, polyethylenes and biodegradable polymers.

The compounds of the subject invention may be administered in a sustained (i.e. controlled) or slow release form. A sustained release preparation is one in which the active ingredient is slowly released within the body of the subject once administered and maintains the desired drug concentration over a minimum period of time. The preparation of sustained release formulations is well understood by persons skilled in the art. Dosage forms may include oral forms, implants and transdermal forms, joint injections, sustained or slow release injectables. For slow release administration, the active ingredients may be suspended as slow release particles or within liposomes, for example.

The compositions herein may be packaged for sale with other active agents or alternatively, other active agents may be formulated with flupirtine or its pharmaceutical salts, derivatives, homologs or analogs thereof and optionally an analgesic agent such as an opioid. The composition may be sold or provided with a set of instructions in the form of a therapeutic protocol. This protocol may also include, in one embodiment, a selection process for type of patient or type of condition or a type of pain.

The present invention further contemplates nanoparticulate formulations which include nanocapsules, nanoparticles, microparticles, liposomes, nanospheres, microspheres, lipid particles, and the like. Such formulations increase the delivery efficacy and bioavailability and reduce the time for analgesic effect of the pain management agents. Nanoparticles generally comprise forms of the agents entrapped within a polymeric framework or other suitable matrix. Nanoparticle formulations are particularly useful for sparingly water soluble drugs. Such formulations also increase bioavailability. One method of formulation is a wet bead milling coupled to a spray granulation.

Thus, a further aspect provides a system for the controlled release of active compounds selected from an omega conotoxin in combination with a neuronal excitation inhibitor or a pharmaceutically acceptable salt, derivative, homolog or analog thereof, alone or together with another analgesic or active agent, wherein the system comprises:

(a) a deposit-core comprising an effective amount of a first active compound and having defined geometric form, and

(b) a support-platform applied to the deposit-core, wherein the support-platform contains a second active compound, and at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

As used herein, the first active substance is one of (i) an omega conotoxin or (ii) a neuronal excitation inhibitor. The second active substance may be (i) or (ii) above.

In another aspect, a system is provided for the controlled release for an omega conotoxin and a neuronal excitation inhibitor, wherein the system comprises:

(a) a deposit-core comprising an effective amount of (1) omega conotoxin and (2) a neuronal excitation inhibitor, the deposit-core having a defined geometric form; and

(b) a support platform applied to the deposit-core, the support platform comprising at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

A further aspect contemplates a system for the controlled release for an omega conotoxin and a sodium channel blocker, wherein the system comprises:

(a) a deposit-core comprising an effective amount of (1) omega conotoxin and (2) a sodium channel blocker, the deposit-core having a defined geometric form; and

(b) a support platform applied to the deposit-core, the support platform comprising at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

Still a further aspect provides a system for the controlled release for an omega conotoxin and a local anaesthetic, wherein the system comprises:

(a) a deposit-core comprising an effective amount of (1) omega conotoxin and (2) a local anaesthetic, the deposit-core having a defined geometric form; and

(b) a support platform applied to the deposit-core, the support platform comprising at least one compound selected from the group consisting of
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

Even yet a further aspect contemplates a system for the controlled release for an omega conotoxin and a modulator of TRPV1 receptor, wherein the system comprises:

(a) a deposit-core comprising an effective amount of (1) omega conotoxin and (2) a modulator of TRPV1 receptor, the deposit-core having a defined geometric form; and

(b) a support platform applied to the deposit-core, the support platform comprising at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

Another aspect provides a system for the controlled release for an omega conotoxin and a modulator of CB2 receptor, wherein the system comprises:

(a) a deposit-core comprising an effective amount of (1) omega conotoxin and (2) a modulator of CB2 receptor, the deposit-core having a defined geometric form; and

(b) a support platform applied to the deposit-core, the support platform comprising at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

The support-platform may comprise polymers such as hydroxypropylmethylcellulose, plasticizers such as a glyceride, binders such as polyvinylpyrrolidone, hydrophilic agents such as lactose and silica, and/or hydrophobic agents such as magnesium stearate and glycerides. The polymer(s) typically make up 30 to 90% by weight of the support-platform, for example about 35 to 40%. Plasticizer may make up at least 2% by weight of the support platform, for example about 15 to 20%. Binder(s), hydrophilic agent(s) and hydrophobic agent(s) typically total up to about 50% by weight of the support platform, for example about 40 to 50%.

The tablet coating may contain one or more water insoluble or poorly soluble hydrophobic excipients. Such excipients may be selected from any of the known hydrophobic cellulosic derivatives and polymers including alkylcellulose, e.g. ethylcellulose, hydroxypropyl cellulose, hydroxypropylmethyl cellulose, carboxymethyl cellulose, and derivatives thereof; polymethacrylic polymers, polyvinyl acetate and cellulose acetate polymers; fatty acids or their esters or salts; long chain fatty alcohols; polyoxyethylene alkyl ethers; polyoxyethylene stearates; sugar esters; lauroyl macrogol-32 glyceryl, stearoyl macrogol-32 glyceryl, and the like. Hydroxypropylmethyl cellulose materials are preferably selected from those low Mw and low viscosity materials such as E-Type methocel, and 29-10 types as defined in the USP.

Other agents or excipients that provide hydrophobic quality to coatings may be selected from any waxy substance known for use as tablet excipients. Preferably they have a HLB value of less than 5, and more preferably about 2. Suitable hydrophobic agents include waxy substances such as carnauba wax, paraffin, microcrystalline wax, beeswax, cetyl ester wax and the like; or non-fatty hydrophobic substances such as calcium phosphate salts, e.g. dibasic calcium phosphate.

The coating may contain a calcium phosphate salt, glyceryl behenate, and polyvinyl pyrollidone, or mixtures thereof, and one or more adjuvants, diluents, lubricants or fillers.

Components in the coating may be as follows, with generally suitable percentage amounts expressed as percentage weight of the coating.

Polyvinyl pyrollidone (Povidone) is preferably present in amounts of about 1 to 25% by weight or the coating, more particularly 4 to 12%, e.g. 6 to 8%.

Glyceryl behenate is an ester of glycerol and behenic acid (a C22 fatty acid). Glyceryl behenate may be present as its mono-, di-, or tri-ester form, or a mixture thereof. Preferably it has an HLB value of less than 5, more preferably approximately 2. It may be present in amounts of about 5 to 85% by weight of the coating, more particularly from 10 to 70% by weight, and in certain preferred embodiments from 30 to 50%.

Calcium phosphate salt may be the dibasic calcium phosphate dihydrate and may be present in an amount of about 10 to 90% by weight of the coating, preferably 20 to 80%, e.g. 40 to 75%.

The coating may contain other common tablet excipients such as lubricants, colourants, binders, diluents, glidants and taste-masking agents or flavourants.

Examples of excipients include colourants such a ferric oxide, e.g. yellow ferric oxide; lubricants such as magnesium stearate; and glidants such as silicon dioxide, e.g. colloidal silicon dioxide. Yellow ferric oxide may be used in amounts of about 0.01 to 0.5% by weight based on the coating; magnesium stearate may be present in amounts of 1 to 20% by weight of the coating, more preferably 2 to 10%, e.g. 0.5 to 1.0%; and colloidal silica may be used in amounts of 0.1 to 20% by weight of the coating, preferably 1 to 10%, more preferably 0.25 to 1.0%.

The core comprises in addition to a drug substance, a disintegrating agent or mixtures of disintegrating agents used in immediate release formulations and well know to persons skilled in the art. The disintegrating agents useful in the exercise of the present invention may be materials that effervesce and or swell in the presence of aqueous media thereby to provide a force necessary to mechanically disrupt the coating material.

A core may contain, in addition to the drug substance, cross-linked polyvinyl pyrollidone and croscarmellose sodium.

The following is a list of contemplated core materials. The amounts are expressed in terms of percentage by weight based on the weight of the core.

Cross-linked polyvinyl pyrollidone is described above and is useful as a disintegrating agent, and may be employed in the core in the amounts disclosed in relation to the core.

Croscarmellose sodium is an internally cross-linked sodium carboxymethyl cellulose (also known as Ac-Di-Sol) useful as a disintegrating agent.

Disintegrating agents may be used in amounts of 5 to 30% by weight based on the core. However, higher amounts of certain disintegrants can swell to form matrices that may modulate the release of the drug substance. Accordingly, particularly when rapid release is required after the lag time it is preferred that the disintegrants is employed in amounts of up to 10% by weight, e.g. about 5 to 10% by weight.

The core may additionally comprise common tablet excipients such as those described above in relation to the coating material. Suitable excipients include lubricants, diluents and fillers, including but not limited to lactose (for example the mono-hydrate), ferric oxide, magnesium stearates and colloidal silica.

Lactose monohydrate is a disaccharide consisting of one glucose and one galactose moiety. It may act as a filler or diluent in the tablets of the present invention. It may be present in a range of about 10 to 90%, preferably from 20 to 80%, and in certain preferred embodiments from 65 to 70%.

The core should be correctly located within the coating to ensure that a tablet has the appropriate coating thickness.

In this way, lag times are reliable and reproducible, and intra-subject and inter-subject variance in bioavailability is avoided. It is advantageous to have a robust control mechanism to ensure that tablets in a batch contain cores having the appropriate geometry in relation to the coating. Controls can be laborious in that they require an operator to remove random samples from a batch and to cut them open to physically inspect the quality of the core (i.e. whether it is intact, and whether it is correctly located). Furthermore, if a significant number of tablets from the sample fail, a complete batch of tablets may be wasted. Applicant has found that if one adds to the core a strong colourant such as iron oxide, such that the core visibly contrasts with the coating when as strong light is shone on the tablet, it is possible for any faults in the position or integrity of the core to be picked up automatically by a camera appropriately located adjacent a tabletting machine to inspect tablets as they are ejected therefrom.

The above formulations also apply to the development of nanoparticle formulations.

Still another aspect provides a composition comprising: (a) an omega conotoxin; and (b) an immediate release neuronal excitation inhibitor.

A method for the delivery of the composition to a subject is provided comprising the step of administering the composition to the subject orally, transdermally, or subdermally, wherein the composition comprises components (a) and (b) as defined above.

In one aspect, a tamper-proof narcotic delivery system is produced which provides for full delivery of narcotic medication and for analgesic action on legitimate patients while at the same time effectively eliminating the problem of tampering by diversion, adulteration, or pulverization of the medication for abuse by addicts. The compositions and methods herein are of value to those practiced in the medical arts and simultaneously possess no value or utility to individuals seeking to abuse or profit from the abuse of such analgesics.

It should be understood that in addition to the ingredients particularly mentioned above, the compositions herein may include other agents conventional in the art, having regard to the type of composition in question. For example, agents suitable for oral administration may include such further agents as binders, sweetners, thickeners, flavouring agents, disintegrating agents, coating agents, preservatives, lubricants and/or time delay agents.

The formulation may also contain carriers, diluents and excipients. Details of pharmaceutically acceptable carriers, diluents and excipients and methods of preparing pharmaceutical compositions and formulations are provided in Remmingtons Pharmaceutical Sciences 18^{th} Edition, 1990, Mack Publishing Co., Easton, Pa., USA.

In an embodiment, the active agents may also be presented for use in veterinary compositions. These may be prepared by any suitable means known in the art. Examples of such compositions include those adapted for:

(a) oral administration, e.g. drenches including aqueous and non-aqueous solutions or suspensions, tablets, boluses, powders, granules, pellets for admixture with feedstuffs, pastes for application to the tongue;

(b) parenteral administration, e.g. subcutaneous, intra-articular, intramuscular or intravenous injection as a sterile solution or suspension or through intra-nasal administration;

(c) topical application, e.g. creams, ointments, gels, lotions, etc.

In another embodiment, the active agents are administered orally, preferably in the form of a tablet, capsule, lozenge or liquid. The administered composition may include a surfactant and/or solubility improver. A suitable solubility improver is water-soluble polyethoxylated caster oil and an example of a suitable surfactant is Cremophor EL. Dose ranges suitable for the omega conotoxin are, for example, 0.01 mg/kg to 10 mg/kg, including, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3.0, 4.0, 5.0, 6.0, 7.0, 8.0, 9.0, 10 mg/kg.

In one aspect, fentanyl is administered at a rate and concentration of 100 micrograms/hour.

In another aspect, tramadol is administered at a rate of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 micrograms/hour or per kg body weight.

In a related aspect an NSAID can be administered at 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 micrograms/hour or per kg body weight.

In a further aspect, a neurosteroid can be administered at 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 micrograms/hour or per kg body weight.

The calcium channel antagonists can be administered without being limited to, a rate of 0.1, 1, 5, 10, 15, 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400 milligrams/hour or per kg body weight.

Mechanical devices are also provided for introduction to or in a body or body cavity coated with a sustained or slow release formulation of an omega conotoxin combined with the neuronal excitation inhibitor. Examples of mechanical devices include stents, catheters, artificial limbs, pins, needles, intrathecal implants and the like. Reference to an “intrathecal implant” includes reference to a cylindrical thread or device comprising a semipermeable membrane which permits passage or partial passage of small molecules (such as nutrients ad drugs in and cellular metabolic products out). The implant may also contain genetically modified or cultured cells (including stem cells) which secrete out useful cytokines and other metabolites. The implant may be designed to release molecules (or intake cellular by-products) for days, weeks, months or even years.

Stents, for example, typically have a lumen, inner and outer surfaces, and openings extending from the outer surface to the inner surface. The present invention extends to a method for coating a surface of a stent. At least a portion of the stent is placed in contact with a coating solution containing a coating material to be deposited on the surface of the stent. A thread is inserted through the lumen of the stent, and relative motion between the stent and the thread is produced to substantially remove coating material within the openings.

The thread can have a diameter substantially smaller than the diameter of the lumen. The thread can be inserted through the lumen either after or prior to contacting the stent with the coating solution. Relative motion between the stent and the thread can be produced prior to contacting the stent with the coating solution to clean the stent. The thread can be either a filament or a cable with a plurality of wires. The thread can be made of a metallic or polymeric material.

The stent can be dipped into the coating solution or spray coated with the coating solution. The coating material can include a biocompatible polymer, either with or without a pharmaceutically active compound.

In one embodiment, the relative motion is oscillatory motion produced by a vibrating device. The oscillations can be changed (magnitude and/or frequency) to vary thickness of the coating solution on the stent. In another embodiment, the relative motion is produced by a shaker table. Regardless of the type of motion, the relative motion can be produced either after or while the stem is in contact with the coating solution.

The relative motion between the stent and the thread can include initially moving the stent in a horizontal direction substantially parallel to the length of the thread and subsequently moving the stent in a vertical direction substantially perpendicular to the length of the thread. The movement in the horizontal direction can be repeated, with pauses between repetitions. The movement in the vertical direction can also be repeated, with the horizontal and vertical movements alternating.

In order to smooth the relative motion, the thread can be coupled to a damping compensator. The damping compensator connects the thread to a vibrating device. In one embodiment, the damping compensator comprises first and second filaments connected to the thread.

The relative motion can be motion of the stent along the thread. For example, a first end of the thread can be attached to a first stand at a first height and a second end of the thread is attached to a second stand at a second height. The relative motion is produced by a gravity gradient, with the first height differing from the second height. Furthermore, the stent can be moved back and forth between the first and second stands by sequentially increasing or decreasing at least one of the first and second heights. In this way, multiple coatings can be applied to the stent.

The relative motion can also be rotation of the stent relative to the thread. A stream of gas can be passed along at least a portion of the surface of the stent to rotate the stent relative to the thread. The rotation can also occur in conjunction with other relative motion between the stent and the thread.

An implantable medical device is also provided having an outer surface covered at least in part by an omega conotoxin and a neuronal excitation inhibitor or pharmaceutically acceptable salts, derivative, homolog or analog thereof and optionally an opioid and/or other active agent, a conformal coating of a hydrophobic elastomeric material incorporating an amount of active material therein for timed delivery therefrom and means associated with the conformal coating to provide a non-thrombogenic surface after the timed delivery of the active material.

In an embodiment, the conformal coating comprises an amount of finely divided biologically active material in the hydrophobic elastomeric material.

The present invention is now described by the following non-limiting examples.

### EXAMPLES

### Example 1

### Systemic, Non-Intrathecal Administration of CVID Induced No Toxicity

In order to determine the safety of systemic, non-intrathecal administration of an omega conotoxin, beagle dogs were continuously infused intravenously (iv) with 80 μg/kg/day for 6 days with CVID.

After 6 days of continuous infusion, no toxicological or pathological effect was observed. In addition, no adverse effects on cardiovascular function was observed.

### Example 2

### Systemic, Non-Intrathecal Administration of CVID has Potent Neuropathic Pain Relieving Capabilities

Using the streptozotocin-induced diabetic neuropathy model of neuropathic pain, omega conotoxins were tested for their analgesic properties when administered alone, or in combination with an inhibitor of neuronal excitation. This diabetic model reproduces the experience of diabetic neuropathic pain in humans (Courteix et al. Pain 53:81-88, 1993). The relative efficacy of CVID or MVIIA (ziconotide) alone or in combination with flupirtine to induce analgesia was assessed.

Male Wistar rats (wt 65-80 g) were used in this model. Animals were housed 5 per cage under standard laboratory conditions. Food and water were provided ad libitum. Rats were injected i.v. with streptozotocin (STZ; Sapphire Bioscience) [150 mg/kg total dose] dissolved in sodium chloride (0.9% w/v). The 150 mg dose was given in two 75 mg/kg injections on consecutive days. Diabetes was confirmed one week after injection of STZ by measurement of tail vein blood glucose levels with Ames Glucofilm test strips and a reflectance colorimeter (Ames Glucometer 3, Bayer Diagnostics). Only animals with final blood glucose levels 15 mM were deemed to be diabetic. The rats were re-tested for hyperglycaemia once per week to confirm continued high blood glucose readings. Hyperalgesia was assessed using the paw pressure test (Randall and Selitto Archiv Inst Pharmacdynamie 111:409, 1957).

Tests took place 5 weeks after the first injection of STZ. Animals that had paw pressure nociceptive thresholds below 30 g (60% of the value in normal weight matched rats) were deemed to have developed hyperalgesia/neuropathic pain and thus used in further experiments.

Animals were then divided into one of the following 10 groups:

1. MVIIA alone (0.02 mg/kg iv bolus; maximum non-sedating dose);
2. CVID alone (0.02 mg/kg iv bolus);
3. CVID alone (2.0 mg/kg iv bolus; maximum non-sedating dose);
4. Flupirtine alone (2.5 mg/kg ip);
5. MVIIA (0.02 mg/kg iv) combined with flupirtine (2.5 mg/kg ip) (maximum non-sedating dose combination);
6. CVID (0.02 mg/kg iv) combined with flupirtine (2.5 mg/kg ip);
7. CVID (0.02 mg/kg iv) combined with flupirtine (5.0 mg/kg ip);
8. Gabapentin alone (50 mg/kg ip);
9. Morphine alone (3.2 mg/kg ip); and
10. Gabapentin (50 mg/kg ip) combined with morphine (3.2 mg/kg ip).

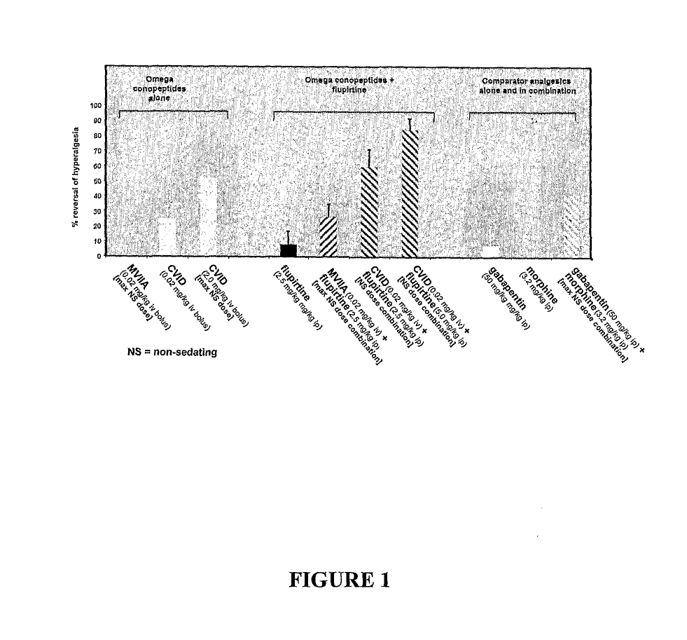

As shown in FIG. 1, MVIIA provided no analgesic effect when administered alone. However, CVID provided significant analgesic effect at both 0.02 mg/kg (approximately 30% reduction in hyperalgesia) and 2.0 mg/kg (greater than 50% reduction in hyperalgesia). The latter result showed improved efficacy over the use of gabapentin or morphine alone or when the two standard pain relievers were used in combination.

Combinations of CVID and flupirtine or MVIIA and flupirtine were also examined for their analgesic effects. As shown in FIG. 1, flupirtine alone provided minimal analgesic effect with a reversal in hyperalgesia of only just over 20%. However, when CVID was combined with flupirtine, reversal of hyperalgesia was significantly improved compared to any of the agents used alone. In particular, CVID (0.02 mg/kg) with flupirtine provided a greater than 60% reversal of hyperalgesia and CVID (2.0 mg/kg) with flupirtine provided an approximately 90% reversal in hyperalgesia.

### Example 3

Systemic, Non-Intrathecal Administration of CVID has Potent Inflammatory Pain Relieving Capabilities

The carrageenan paw inflammation model involves induction of inflammation and oedema in one paw of the rat by the intraplantar injection of carrageenan (6 mg per 150 μl). This is a single intraplantar injection using a fine needle and syringe whilst restraining the rat gently. The rats were then subjected to nociceptive threshold measurement using withdrawal from stimulation with Von Frey hairs (measures allodynia). Nociceptive thresholds were measured in groups of rats prior to the intraplantar injection. The measurements were continued three hours after the intraplantar injection when the inflammation had developed. At that stage allodynia had developed. Rats were then treated i.p. with CVID alone at 20 mg/kg or 50 mg/kg or using a saline control. Measurements of the allodynia nociceptive thresholds were then monitored and used to assess the antinocicepetive effect of the CVID.

CVID was effective at inducing an analgesic response when administered alone when compared to saline administration.

These antinociceptive responses were observed for 4-6 hours following injection of the CVID bolus.

### Example 4

### Systemic, Non-Intrathecal Administration of CVID in Bone Cancer Model

### Rat Model of Bone Cancer Pain

Sprague-Dawley rats receive intra-tibial injections of syngeneic MRMT-1 rat mammary gland carcinoma cells and develop behavioural signs indicative of pain, including: mechanical allodynia, difference of weight bearing between hind paws and mechanical hyperalgesia. The development of the bone tumour and structural damage to the bone is monitored by radiological analysis, quantitative measurement of mineral content and histology. Intra-tibial injections of 3×10³ or 3×10⁴ syngeneic MRMT-1 cells produce a rapidly expanding tumor within the boundaries of the tibia, causing severe remodelling of the bone. Radiographs show extensive damage to the cortical bone and the trabeculae by day 10-14 after inoculation of 3×10³ MRMT-1 cells, and by day 20, the damage is threatening the integrity of the tibial bone. While both mineral content and mineral density decrease significantly in the cancerous bone, osteoclast numbers in the peritumoural compact bone remain unchanged. Tartarate-resistant acid phosphatase staining reveals a large number of polykariotic cells, resembling those of osteoclasts within the tumor. No tumor growth is observed after the injection of heat-killed MRMT-1 cells. Intra-tibial injections of 3×10³ or 3×10⁴ MRMT-1 cells, heat-killed cells or vehicle do not show changes in body weight and core temperature over 19-20 days. The general activity of animals after injection with live or heat-killed MRMT-1 cells is higher than that of the control group rats which receive intra-tibial injections of MRMT-1 cells display the gradual development of mechanical allodynia and mechanical hyperalgesia and reduce weight bearing on the affected limb, beginning on day 12-14 or 10-12 following injection of 3×10³ or 3×10⁴ cells, respectively. These symptoms are not observed in rats receiving heat-killed cells or vehicle.

Experimental and control animals are each divided into three groups 1, 2, 3, wherein animal will receive either omega conotoxin alone or in combination with a neuronal excitation inhibitor.

### Example 5

### Antinociceptive Potencies of Leconotide and Ziconotide

Intravenous administration [iv] of leconotide is less toxic than ziconotide (Wright et al. British Journal of Pharmacology 131:1325-36, 2000). This example compares the antinociceptive potencies of leconotide and ziconotide given iv alone and in combinations with a KCNQ potassium channel opener flupirtine [intraperitoneally; ip], in a rat model of diabetic neuropathic pain.

322 rats were given streptozotocin [STZ; 150 mg/kg ip] to cause diabetic neuropathy and hyperalgesia (Courteix et al. 1993 supra). All subsequent experiments were performed on these rats with ≧30% hyperalgesia to noxious heat (Hargreaves et al. Pain 32:77-88, 1988) measured by an observer unaware of the treatment given to each rat. 112 such rats were given injections of each conopeptide iv alone and in combinations with flupirtine ip as well as saline controls and placed in an open field activity monitor to define the maximum non-sedating doses and dose combinations. A range of doses up to the maximum non-sedating doses and dose combinations were then given to 210 rats with hyperalgesia. Dose response and 3D surface plots were constructed.

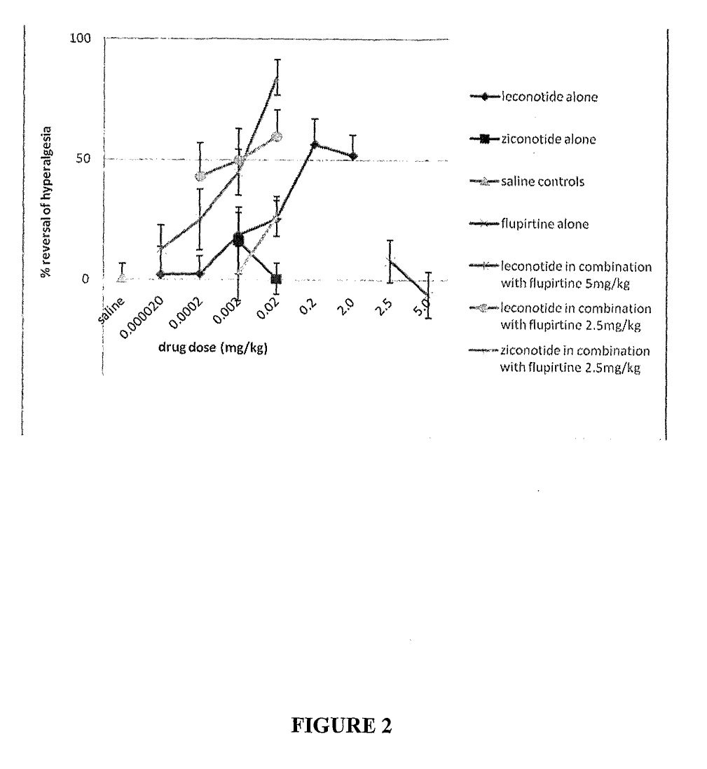

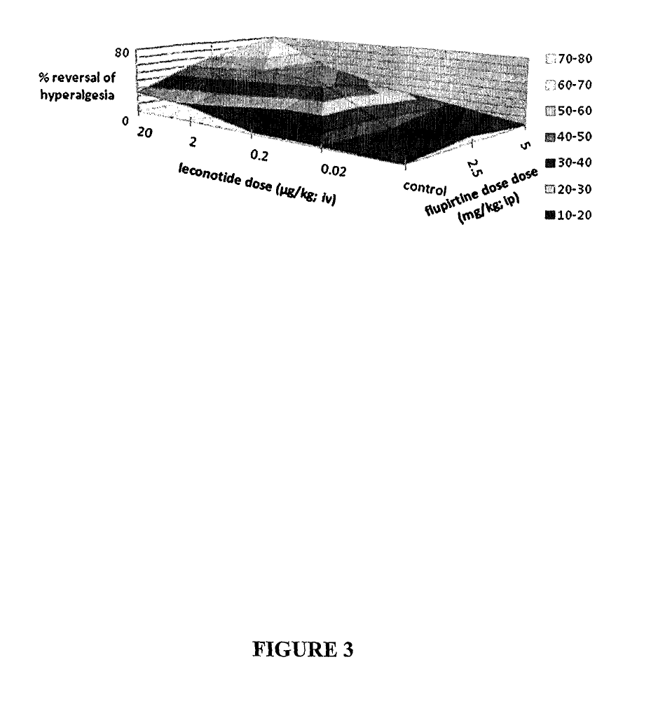

The maximum non-sedating dose of leconotide [2 mg/kg iv; Table 1] caused 51.7% reversal of hyperalgesia compared with 0.4% reversal for the highest non-sedating dose of ziconotide [0.02 mg/kg iv; p<0.001, one-way ANOVA; FIG. 2]. Leconotide caused dose related antinociceptive effects that were potentiated by coadministration with flupirtine which was ineffective when given alone in this model [FIGS. 2-4]. Leconotide [0.02 mg/kg] and flupirtine [2.5 mg/kg] given alone caused 25.3±7.6 and 8±8% reversal of hyperalgesia when given alone but in combination they caused 59.5±11.2% reversal of hyperalgesia [p<0.01; one-way ANOVA].

These results indicate that leconotide has a wider clinical application than ziconotide because antinociception can be achieved with leconotide without the necessity for intrathecal administration.

TABLE 1

| | mean rest | | |
|---|---|---|---|
| ASSESSMENT OF SEDATION: | times | | |
| open field activity monitor | (seconds | SEM | n |
| saline controls | 943.79 | 12.40 | 29 |
| drugs given alone | | | |
| leconotide 2 mg/kg iv | 982.89 | 26.96 | 9 |
| ziconotide 0.2 mg/kg iv | 1033.00 | 18.59 | 8 |
| ziconotide 0.02 mg/kg iv | 993.13 | 16.86 | 8 |
| flupirtine 2.5 mg/kg ip | 943.83 | 55.51 | 6 |
| combinations | | | |
| flupirtine 5 mg/kg ip + leconotide 0.2 mg/kg iv | 1091.13*** | 10.70 | 8 |
| flupirtine 5 mg/kg ip + leconotide 0.02 mg/kg iv | 1040.75 | 28.21 | 5 |
| flupirtine 5 mg/kg ip + leconotide 0.002 mg/kg iv | 1050.00 | 30.58 | 5 |
| flupirtine 2.5 mg/kg ip + leconotide 0.2 mg/kg iv | 1058.33* | 18.01 | 6 |
| flupirtine 2.5 mg/kg ip + leconotide 0.02 mg/kg iv | 1032.50 | 27.18 | 6 |
| flupirtine 10 mg/kg ip + ziconotide 0.02 mg/kg iv | 1100.63*** | 12.88 | 8 |
| flupirtine 5 mg/kg ip + ziconotide 0.02 mg/kg iv | 1030.86 | 31.45 | 7 |
| flupirtine 2.5 mg/kg ip + ziconotide 0.02 mg/kg iv | 1002.86 | 23.53 | 7 |

*p < 0.05;
**p < 0.01;
***p < 0.001;
One way ANOVA, Tukey post hoc comparison with saline controls

### Example 6

### Isobolographic Analysis of the Interaction Between Flupirtine and Morphine in Antinociception in a Rat Model of Prostate Bone Cancer Pain

Current treatments for cancer pain are often inadequate, particularly when metastasis to bone is involved. Many pains are resistant to morphine so that the dose has to be increased causing side effects that reduce the quality of life. The addition of another drug to the treatment regimen that has a complementary analgesic effect may increase the overall analgesia without the necessity to increase doses avoiding dose-related opioid side effects. This project investigated the synergistic effect of the addition of the potassium channel modulator, flupirtine to morphine in causing antinociception in a rat model of prostate bone cancer pain.

Male Wistar rats (HsdBrlHan WIST strain, 100-200 g; n=123) were used. They were anaesthetized and syngeneic prostate cancer cells (AT3B-1 prostate cancer; ATCC, VA, USA) were injected into the medullary cavity of the tibia as described previously (Rui-Xin Zhang et al. Pain 118:125-36, 2005). This led to expanding tumor within the bone 2-3 weeks later, together with the concurrent development of hyperalgesia to heat applied to the ipsilateral hind paw. The maximum non-sedating doses of morphine and flupirtine given alone and in combinations were defined using an open field activity monitor [Table 2]. Paw withdrawal thresholds from noxious heat were measured as described by Hargreaves et al. 1988 supra. Dose response curves for morphine (0.13-10 mg/kg ip) and flupirtine (1.25-10 mg/kg ip) given alone and in fixed dose combinations were plotted and subjected to an isobolographic analysis as described by Tallarida R J Pain 49:93-7, 1992 [Table 3].

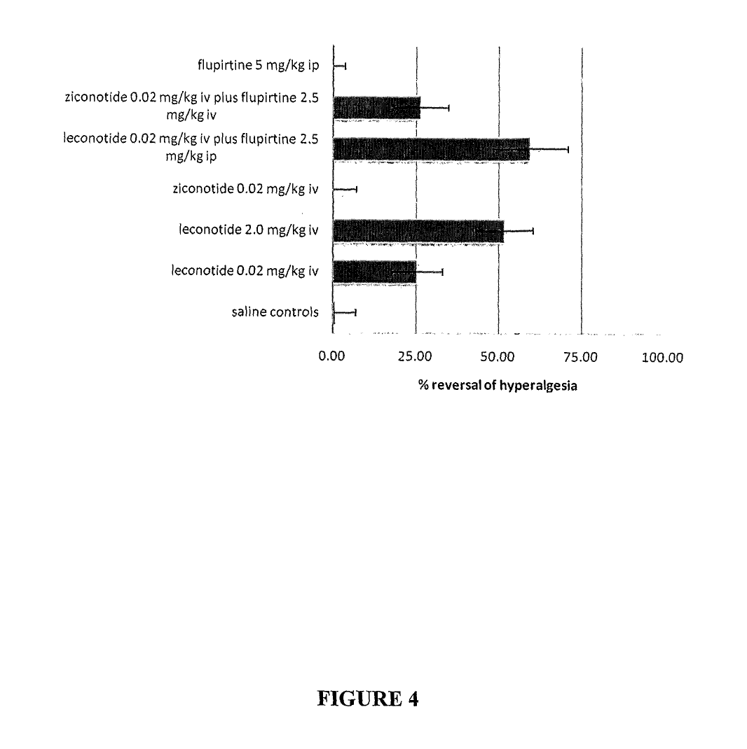

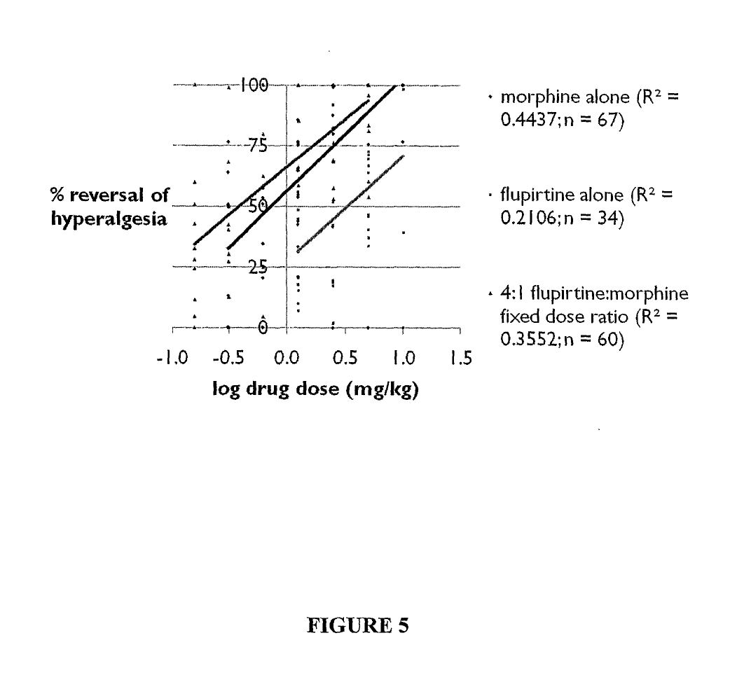

Non-sedating doses of both morphine (ED50=0.735 mg/kg) and flupirtine (ED50=3.317 mg/kg) caused dose related antinociception [FIG. 4]. Isobolographic analysis revealed that there was a synergistic interaction between flupirtine and morphine [Table 3; FIG. 5].

These results indicate that flupirtine in combination with morphine is useful clinically in the management of pain caused by bone cancer to provide better analgesia at lower morphine doses.

TABLE 2
Test for sedation: open field activity monitor

| treatment | mean | SEM | n |
|---|---|---|---|
| saline control | 776.0 | 14.0 | 33 |
| morphine 20 mg/kg | 937.4 | 22.9 | 12 |
| morphine 10 mg/kg | 814.2 | 27.8 | 10 |
| morphine 5 mg/kg | 752.6 | 37.3 | 9 |
| gabapentin 200 mg/kg | 886.5 | 48.0 | 12 |
| gabapentin 100 mg/kg | 817.1 | 56.3 | 10 |
| flupirtine 16 mg/kg | 923.5 | 27.1 | 10 |
| flupirtine 8 mg/kg | 840.1 | 13.6 | 15 |
| flupirtine 8 mg/kg + morphine 2 mg/kg | 868.2 | 22.4 | 14 |
| flupirtine 4 mg/kg + morphine 1 mg/kg | 814.2 | 17.9 | 15 |

figures in red = treatment caused sedation compared with saline controls p < 0.01; one way ANOVA with Dunnett post hoc correction

TABLE 3
ISOBOLOGRAM

| ED₅₀ | | Mean | SEM |
|---|---|---|---|
| Flupirtine alone from DR curve | | 3.317 | 0.876 |
| Morphine alone from DR curve | | 0.735 | 0.158 |
| 4:1 mix | Flupirtine | 1.559 | 0.263 |
| Flupirtine:Morphine | Morphine | 0.390 | 0.066 |
| Calculated | | | |
| 4:1 mix | Flupirtine | 0.312 | 0.076 |
| Flupirtine:Morphine | Morphine | 0.078 | 0.019 |
| From DR curve | | | |

### Example 7

### Interaction Between Leconotide and Morphine in Antinociception in a Rat Model of Prostate Bone Cancer Pain

Using the same prostate bone cancer model as in Example 6 the antinociceptive effect of morphine and leconotide given alone and in combinations was assessed for non sedating doses and dose combinations. Table 4 shows the results of the sedation tests using open field activity monitoring in rats with intratibial cancer. The figures in red indicate sedating doses and dose combinations excluded from the study of antinociceptive effects.

TABLE 4

| | mean | SEM | n |
|---|---|---|---|
| saline iv + saline ip | 775.2 | 16.4 | 25 |
| Leconotide 200 μg/kg iv + saline ip | 886.1 | 25.0 | 10 |
| Leconotide 20 μg/kg iv + saline ip | 819.7 | 22.2 | 12 |
| Leconotide 200 μg/kg iv + Morphine | 963.5 | 21.2 | 10 |
| 2.5 mg/kg ip | | | |
| Leconotide 200 μg/kg iv + Morphine | 943.8 | 26.8 | 10 |
| 1.25 mg/kg ip | | | |
| Leconotide 20 μg/kg iv + Morphine | 791.4 | 21.7 | 10 |
| 2.5 mg/kg ip | | | |
| saline control IP | 776.03 | 13.97 | 33 |
| morphine 10 mg/kg ip | 814.20 | 27.85 | 10 |
| morphine 20 mg/kg ip | 937.42 | 22.86 | 12 |
| morphine 5 mg/kg ip | 752.56 | 37.26 | 9 |
| gabapentin 100 mg/kg ip | 817.10 | 56.30 | 10 |
| gabapentin 200 mg/kg ip | 886.50 | 48.02 | 12 |
| flupirtine 16 mg/kg ip | 923.50 | 27.14 | 10 |
| flupirtine 8 mg/kg + morphine 2 mg/kg ip | 868.21 | 22.42 | 14 |
| flupirtine 8 mg/kg ip | 840.13 | 13.58 | 15 |
| flupirtine 4 mg/kg + morphine 1 mg/kg ip | 814.20 | 17.92 | 15 |

figures in bold indicate significant sedation compared with saline controls

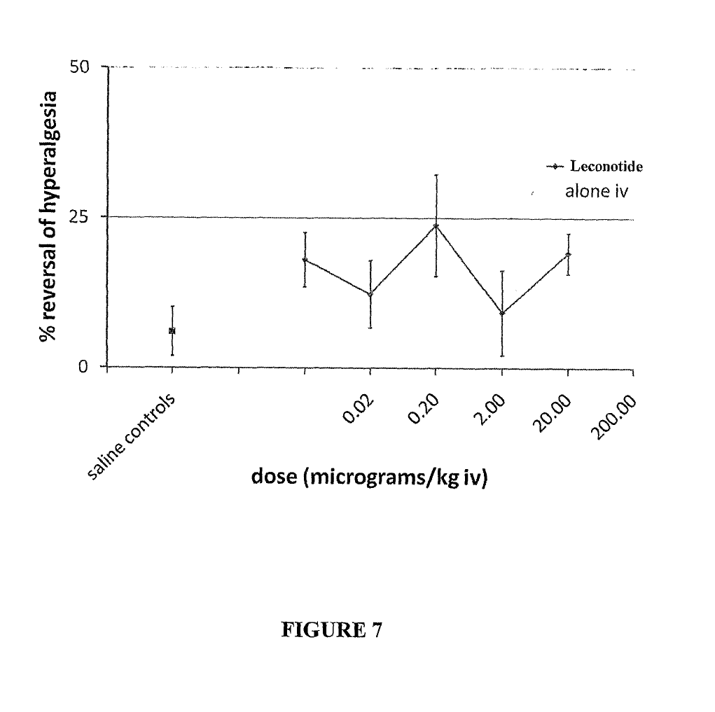

FIG. 7 shows the dose response curve for leconotide given alone. Leconotide alone caused small, statistically non-significant reversal of the hyperalgesia caused by intratibial prostate bone cancer (p>0.05—one way ANOVA with Dunnett's post hoc correction.

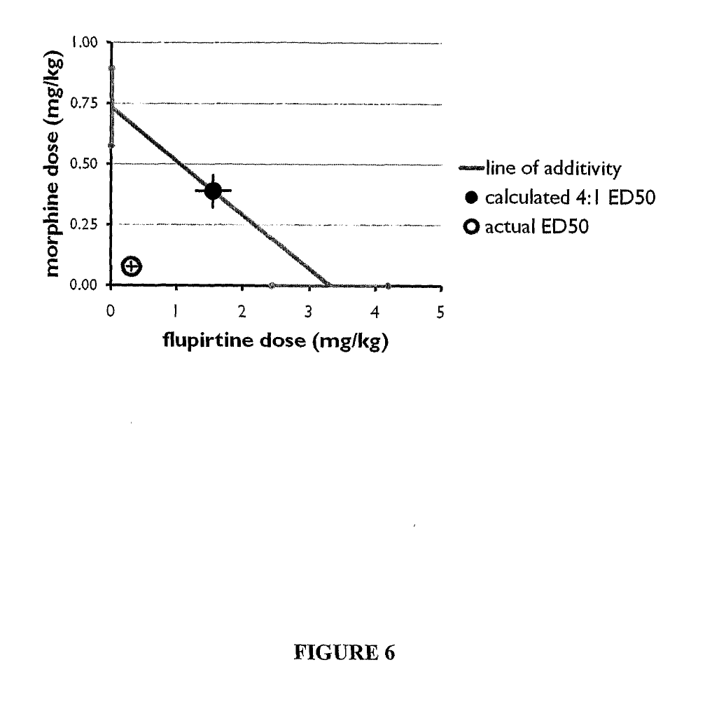

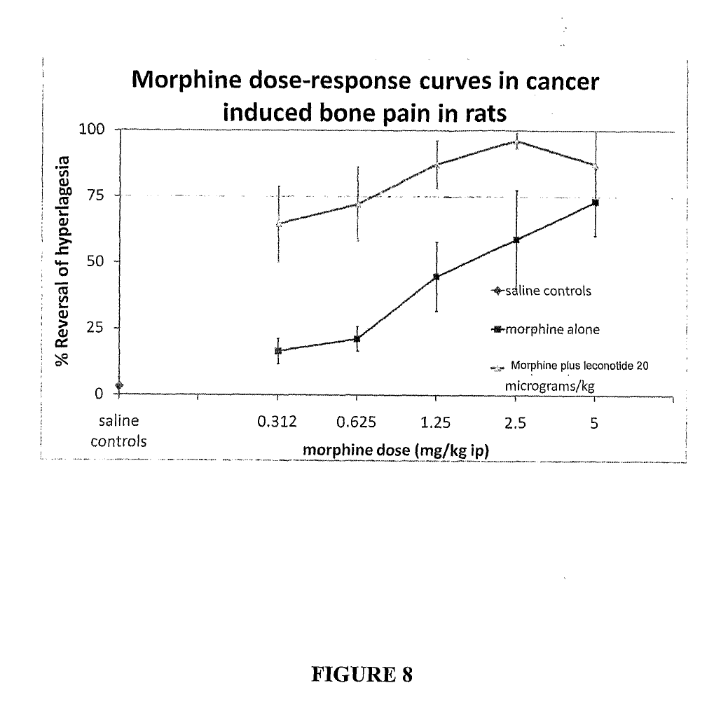

FIG. 8 shows the dose response curves for morphine alone and in combination with leconotide at the dose of 20 micrograms/kg given intravenously. Morphine caused dose related antinociceptive effects that reached 73% reversal of hyperalgesia at the maximum non-sedating dose of 5 mg/kg ip. Coadministration of leconotide 20 mcg/kg (a dose that caused no significant antinociceptive effects when given alone—see FIG. 6) caused a significant leftward shift of the morphine dose response curve. Leconotide potentiates morphine antinociception in this rate bone cancer model.

Those skilled in the art will appreciate that the invention described herein is susceptible to variations and modifications other than those specifically described. It is also to be understood that the invention includes all such variations and modifications. The present invention also includes all steps, features, compositions and compounds referred to, or indicated in this specification, individually or collectively, and any and all combinations of any two or more of said steps or features.

### BIBLIOGRAPHY

Bundgaard Design of Prodrugs, Elsevier 1985
Courteix et al. Pain 53:81-88, 1993
Grimwood et al. Molecular Pharmacology 4:923 1992
Hargreaves et al. Pain 32:77-88, 1988
Jacobson et al. J Pharmacol Exp Ther 110:243, 1987
Leeson, P. D. Drug Design for Neuroscience 13:338-381, 1993
Mayer et al. J Neurophysiol 645, 1988.
Olney & Farber Neuropsychopharmacology 13:335, 1995,
Randall and Selitto Archiv Inst Pharmacdynamie 111:409, 1957
Remmingtons Pharmaceutical Sciences 18^{th} Edition, 1990, Mack Publishing Co., Easton, Pa., USA
Rui-Xin Zhang et al. Pain 118:125-36, 2005
Sawynok et al. Pain 80:45, 1999
Sawynok et al. Pain 82:149, 1999
Tallarida R J Pain 49:93-7, 1992
Thurkauf et al. J Med Chem 31:2257, 1988
Wright et al. British Journal of Pharmacology 131:1325-36, 2000
Yoneda et al. J Neurochem 62:102, 1994

1. A method for inducing an analgesic response to pain in a subject, said method comprising the systemic, non-intrathecal administration to said subject of an amount of an omega conotoxin effective in reducing the level of or otherwise ameliorating the sensation of pain.

2. The method of claim 1 further comprising the administration of a neuronal excitation inhibitor.

3. The method of claim 1 or 2 wherein the pain is neuropathic pain or nociceptive pain.

4. The method according to claim 1 or 2 or 3, wherein the analgesic response is induced without causing overt sedation.

5. The method of claim 4 wherein the omega conotoxin is selected from the group consisting of CVID, GVIA, MVIIA and SNX-111.

6. The method of claim 4 wherein the neuronal excitation inhibitor is flupirtine or a pharmaceutically acceptable salt thereof.

7. The method of claim 4 wherein the neuronal excitation inhibitor is retigabine or a pharmaceutically acceptable salt thereof.

8. The method of claim 4 wherein the neuronal excitation inhibitor is a potassium channel opener.

9. The method of claim 4 wherein the neuronal excitation inhibitor is an opioid or is a pharmaceutically acceptable salt, derivate, homolog or analog thereof.

10. The method of claim 4 wherein the neuronal excitation inhibitor is an NMDA antagonist.

11. The method of claim 4 wherein the neuronal excitation inhibitor is modulator of TRPV1 receptor.

12. The method of claim 6 or 7 wherein flupirtine or retigabine is administered in an amount of about 0.5 μg to about 20 mg per kg of body weight.

13. The method of claim 4 wherein the subject is a human.

14. A delivery system for inducing an analgesic response in a subject having pain, said delivery system comprising combined or separate formulations of (1) an omega conotoxin; (2) an neuronal excitation inhibitor; and optionally (3) one or more further active agents.

15. The delivery system of claim 14 wherein the omega conotoxin is selected from the group consisting of CVID, GVIA, MVIIA and SNX-111.

16. The delivery system of claim 14 wherein the neuronal excitation inhibitor is flupirtine or a pharmaceutically acceptable salt thereof.

17. The delivery system of claim 14 wherein the neuronal excitation inhibitor is retigabine or a pharmaceutically acceptable salt thereof.

18. The delivery system of claim 14 wherein the neuronal excitation inhibitor is a potassium channel opener.

19. The delivery system of claim 14 wherein the neuronal excitation inhibitor is an opioid or a pharmaceutically acceptable salt, derivative, homolog or analog thereof.

20. The delivery system of claim 14 where in the neuronal excitation inhibitor is an NMDA antagonist.

21. The delivery system of claim 14 wherein the neuronal excitation inhibitor is a calcium channel antagonist.

22. The delivery system of claim 14 wherein the neuronal excitation inhibitor is an NSAID.

23. The delivery system of claim 14 wherein the neuronal excitation inhibitor is a modulator of TRPV1 receptor.

24. The delivery system of claim 16 or 17 wherein flupirtine or retigabine is administered in an amount of about 0.5 μg to about 20 mg per kg of body weight.

25. A method of treating pain associated with a disease or physiological condition in a subject, said method comprising administering to said subject an effective amount of an omega conotoxin.

26. The method of claim 25 further comprising the administration of a neuronal excitation inhibitor.

27. The method according to claim 25 or 26, wherein overt sedation is not induced.

28. The method of claim 27 wherein the omega conotoxin is selected from the group consisting of CVID, GVIA, MVIIA and SNX-111.

29. The method of claim 27 wherein the neuronal excitation inhibitor is flupirtine or a pharmaceutically acceptable salt thereof.

30. The method of claim 27 wherein the neuronal excitation inhibitor is retigabine or a pharmaceutically accepted salt thereof.

31. The method of claim 27 wherein the neuronal excitation inhibitor is a potassium channel opener.

32. The method of claim 27 wherein the neuronal excitation inhibitor is an opioid or a pharmaceutically acceptable salt, derivative, homolog or analog thereof.

33. The method of claim 27 where in the neuronal excitation inhibitor is an NMDA antagonist.

34. The method of claim 27 wherein the neuronal excitation inhibitor is a calcium channel antagonist.

35. The method of claim 27 wherein the neuronal excitation inhibitor is an NSAID.

36. The method of claim 27 wherein the neuronal excitation inhibitor is a sodium channel blocker.

37. The method of claim 27 wherein the neuronal excitation inhibitor is a modulator of TRPV1 receptor.

38. The method of claim 29 or 30 wherein flupirtine or retigabine is administered in an amount of about 0.5 μg to about 20 mg per kg of body weight.

39. A system for the controlled release of active compounds selected from an omega conotoxin and a neuronal excitation inhibitor or a pharmaceutically acceptable salt, derivative, homolog or analog thereof, wherein the system comprises:
(a) a deposit-core comprising an effective amount of a first active compound and having defined geometric form, and
(b) a support-platform applied to the deposit-core, wherein the support-platform contains a second active compound, and at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

40. A system for the controlled release for an omega conotoxin and a neuronal excitation inhibitor wherein the system comprises:
(a) a deposit-core comprising an effective amount of (1) an omega conotoxin and (2) a neuronal excitation inhibitor form; and
(b) a support platform applied to the deposit-core, the support platform comprising at least one compound selected from the group consisting of:
(i) a polymeric material which swells on contact with water or aqueous liquids and a gellable polymeric material wherein the ratio of the swellable polymeric material to the gellable polymeric material is in the range 1:9 to 9:1, and
(ii) a single polymeric material having both swelling and gelling properties, and wherein the support-platform is an elastic support applied to the deposit-core so that it partially covers the surface of the deposit-core and follows changes due to hydration of the deposit-core and is slowly soluble and/or slowly gellable in aqueous fluids.

41. A system for the controlled release of claim 39 or 40 wherein the support platform comprises a hydroxypropylmethyl cellulose.

42. A system for the controlled release of claim 39 or 40 wherein the support platform comprises a plasticizer, a binder, a hydrophilic agent and a hydrophobic agent.

43. A method of treatment of a subject said method comprising selecting a subject on the basis of symptoms of pain and administering to said subject an omega conotoxin and a neuronal excitation inhibitor wherein the treatment does not cause overt sedation.

44. The method of claim 43 wherein the pain is neuropathic pain or nociceptive pain.

45. The method of claim 43 or 44 wherein the subject is a human.
